(19) United States
(12) Patent Application Publication
(10) Pub. No.: US 2004/0044032 A1
(43) Pub. Date: Mar. 4, 2004

(54) Farnesyl protein transfer inhibitors for the treatment of inflammatory bowel disease

(76) Inventors: David William End, Ambler, PA (US); Charles Ronald Bowden, North Wales, PA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/433,243

(22) PCT Filed: Nov. 20, 2001
(86) PCT No.: PCT/EP01/13540

(51) Int. Cl. A61K 31/4709

(52) U.S. Cl. 514/312; 514/314

(57) ABSTRACT

The present invention relates to the use of certain farnesyl protein transferase inhibitors for the manufacture of a medicament for the treatment of inflammatory bowel disease.

The present invention relates to the new use of certain farnesyl protein transferase inhibitors in the manufacture of a medicament for the treatment of inflammatory bowel disease.

Inflammatory bowel disease (IBD) identifies a group of chronic inflammatory disorders involving the (mainly) lower gastrointestinal tract. Although the exact aetiology of these disorders remains elusive, enviromental, genetic and immunological factors are implicated. Inflammatory bowel diseases such as Crohn's disease and ulcerative colitis are chronic progressive inflammatory conditions, with high morbidity, and potentially life-threatening complications (e.g. toxic megacolon, increased risk for colon cancer).

Ulcerative colitis involves an inflammatory reaction affecting the colon. The colon appears ulcerated and hemorrhaged. The inflammation is generally uniform and continuous and often involves the rectum. The major symptoms of ulcerative colitis include bloody diarrhea or constipation, abdominal pain, signs of dehydration, anemia, fever and weight loss. Extracolonic manifestations may include arthritis, evidence of liver disease, skin disease and lung disease.

Crohn's disease is characterized by inflammation extending through all layers of the intestional wall and often including the mesenteric lymph nodes. The inflammations may penetrate the mucosa and coalesce to form channels called fistulas and fissures. In Crohn's disease inflammations of the bowel are often discontinous and often granulomatous (see Horrison's Priciples of Internal Medicine, thirteenth ed., 1994, by McGraw-Hill, Inc. ISBN 0-07-032370-4, pg., 1403-1416).

Current therapies include but are not limited to treatment with aminosalicylates (e.g. sulfasalazine, olsalazine), corticosteroids (prednisolone, budenoside), and immuno-modulating drugs (azathioprine, 6-MP). Molecules which target specific steps in the inflammatory cascade, have been recently introduced or are under investigation for the treatment of IBD. However, approximately 25% of IBD patients will require surgery (colectomy) during the course of the disease. Surgical procedures are expensive, and associated with increased mortality and morbidity. There is undoubtedly a need for effective treatment of IBD.

It is, therefore, an object of the present invention to provide a new method for the treatment of inflammatory bowel disease.

WO-97/21701 describes the preparation, formulation and pharmaceutical properties of farnesyl protein transferase inhibiting (imidazoly-5-yl)methyl-2-quinolinone derivatives of formulae (I), (II) and (III), as well as intermediates of formula (II) and (III) that are metabolized in vivo to the compounds of formula (I). The compounds of formulas (I), (II) and (III) are represented by

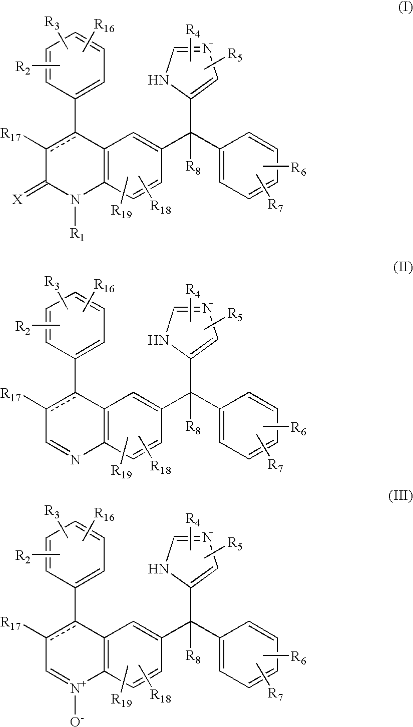

the pharmaceutically acceptable acid or base addition salts and the stereochemically isomeric forms thereof, wherein

the dotted line represents an optional bond;

X is oxygen or sulfur;

R¹ is hydrogen, C₁₋₁₂alkyl, Ar¹, Ar²C₁₋₆alkyl, quinolinylC₁₋₆alkyl, pyridylC₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl, or a radical of formula -Alk¹—C(═O)—R⁹, -Alk¹—S(O)—R⁹ or -Alk¹—S(O)₂—R⁹, whereinAlk¹ is C₁₋₆alkanediyl,

R⁹ is hydroxy, C₁₋₆alkyl, C₁₋₆alkyloxy, amino, C₁₋₈alkylamino or C₁₋₈alkylamino substituted with C₁₋₆alkyloxycarbonyl;

R², R³ and R¹⁶ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, aminoC₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl, 4,4-dimethyloxazolyl; or

when on adjacent positions R² and R³ taken together may form a bivalent radical of formula

—O—CH₂—O— (a-1),

—O—CH₂—CH₂—O— (a-2),

—O—CH═CH— (a-3),

—O—CH₂—CH₂— (a4),

—O—CH₂—CH₂—CH₂— (a-5), or

—CH═CH—CH═CH— (a-6);

R⁴ and R⁵ each independently are hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;

R⁶ and R⁷ each independently are hydrogen, halo, cyano, C₁₋₆alkyl, C₁₋₆alkyloxy, Ar²oxy, trihalomethyl, C₁₋₆alkylthio, di(C₁₋₆alkyl)amino, or when on adjacent positions R⁶ and R⁷ taken together may form a bivalent radical of formula

—O—CH₂—O— (c-1), or

—CH═CH—CH═CH— (c-2);

R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, carboxyC₁₋₆alkyl, hydroxyC₁₋₆alkyl, aminoC₁₋₆alkyl, mono- or di(C₁₋₆alkyl)aminoC₁₋₆alkyl, imidazolyl, haloC₁₋₆alkyl, C₁₋₆alkyloxyC₁₋₆alkyl, aminocarbonylC₁₋₆alkyl, or a radical of formula

—O—R¹⁰ (b-1),

—S—R¹⁰ (b-2),

—N—R¹¹R¹² (b-3),

wherein R¹⁰ is hydrogen, C₁₋₆alkyl, C₁₋₆alkylcarbonyl, Ar¹, Ar²C₁₋₆alkyl, C₁₋₆alkyloxycarbonylC₁₋₆alkyl, or a radical or formula -Alk²-OR¹³ or -Alk²-NR¹⁴R¹⁵;

R¹¹ is hydrogen, C₁₋₁₂alkyl, Ar¹ or Ar²C₁₋₆alkyl;

R¹² is hydrogen, C₁₋₆alkyl, C₁₋₁₆alkylcarbonyl, C₁₋₆alkyloxycarbonyl, C₁₋₆alkylaminocarbonyl, Ar¹, Ar²C₁₋₆alkyl, C₁₋₆alkylcarbonylC₁₋₆alkyl, a natural amino acid, Ar¹carbonyl, Ar²C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical or formula -Alk²-OR¹³ or -Alk²-NR¹⁴R¹⁵;

wherein Alk² is C₁₋₆alkanediyl;

R¹³ is hydrogen, C₁₋₆alkyl, C₁₋₆alkylcarbonyl, hydroxyC₁₋₆alkyl, Ar¹ or Ar²C₁₋₆alkyl;

R¹⁴ is hydrogen, C₁₋₆alkyl, Ar¹ or Ar²C₁₋₆alkyl;

R¹⁵ is hydrogen, C₁₋₆alkyl, C₁₋₆alkylcarbonyl, Ar¹ or Ar²C₁₋₆alkyl;

R¹⁷ is hydrogen, halo, cyano, C₁₋₆alkyl, C₁₋₆alkyloxycarbonyl, Ar¹;

R¹⁸ is hydrogen, C₁₋₆alkyl, C₁₋₆alkyloxy or halo;

R¹⁹ is hydrogen or C₁₋₆alkyl;

Ar¹ is phenyl or phenyl substituted with C₁₋₆alkyl, hydroxy, amino, C₁₋₆alkyloxy or halo; and

Ar² is phenyl or phenyl substituted with C₁₋₆alkyl, hydroxy, amino, C₁₋₆alkyloxy or halo.

WO-97/16443 concerns the preparation, formulation and pharmaceutical properties of farnesyl protein transferase inhibiting compounds of formula (IV), as well as intermediates of formula (V) and (VI) that are metabolized in vivo to the compounds of formula (IV). The compounds of formulas (IV), (V) and (VI) are represented by

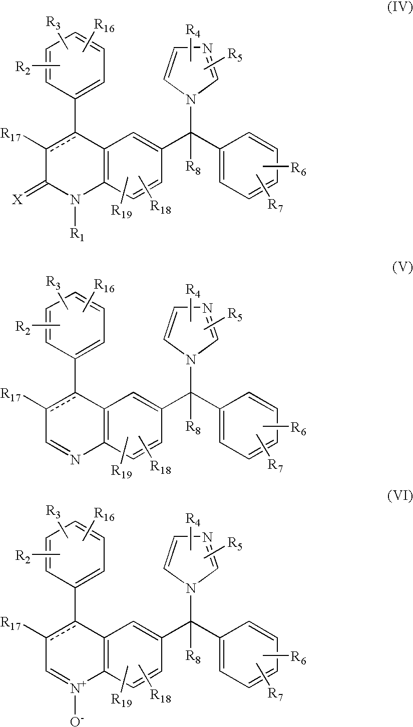

the pharmaceutically acceptable acid or base addition salts and the stereochemically isomeric forms thereof, wherein

the dotted line represents an optional bond;

X is oxygen or sulfur;

R¹ is hydrogen, C₁₋₁₂alkyl, Ar¹, Ar²C₁₋₆alkyl, quinolinylC₁₋₆alkyl, pyridyl-C₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl, or a radical of formula -Alk¹-C(═O)—R⁹, -Alk¹-S(O)—R⁹ or -Alk¹-S(O)₂—R⁹, whereinAlk¹ is C₁₋₆alkanediyl,

R⁹ is hydroxy, C₁₋₆alkyl, C₁₋₆alkyloxy, amino, C₁₋₈alkylamino or C₁₋₈alkylamino substituted with C₁₋₆alkyloxycarbonyl;

R² and R³ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, aminoC₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl; or

when on adjacent positions R² and R³ taken together may form a bivalent radical of formula

—O—CH₂—O— (a-1),

—O—CH₂—CH₂—O— (a-2),

—O—CH═CH— (a-3),

—O—CH₂—CH₂— (a4),

—O—CH₂—CH₂—CH₂ (a-5), or

—CH═CH—CH═CH— (a-6);

R⁴ and R⁵ each independently are hydrogen, Ar¹, C₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;

R⁶ and R⁷ each independently are hydrogen, halo, cyano, C₁₋₆alkyl, C₁₋₆alkyloxy or Ar²oxy;

R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, hydroxycarbonylC₁₋₆alkyl, hydroxyC₁₋₆alkyl, aminoC₁₋₆alkyl, mono- or di(C₁₋₆alkyl)aminoC₁₋₆alkyl, haloC₁₋₆alkyl, C₁₋₆alkyloxyC₁₋₆alkyl, aminocarbonylC₁₋₆alkyl, Ar¹, Ar²C₁₋₆alkyloxyC₁₋₆alkyl, C₁₋₆alkylthioC₁₋₆alkyl;

R¹⁰ is hydrogen, C₁₋₆alkyl, C₁₋₆alkyloxy or halo;

R¹¹ is hydrogen or C₁₋₆alkyl;

Ar¹ is phenyl or phenyl substituted with C₁₋₆alkyl, hydroxy, amino, C₁₋₆alkyloxy or halo;

Ar² is phenyl or phenyl substituted with C₁₋₆alkyl, hydroxy, amino, C₁₋₆alkyloxy or halo.

WO-98/40383, concerns the preparation, formulation and pharmaceutical properties of farnesyl protein transferase inhibiting compounds of formula (VII)

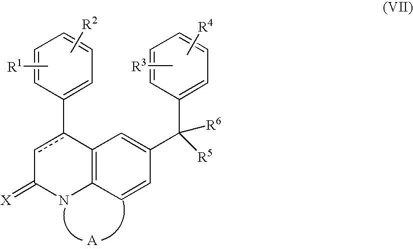

the pharmaceutically acceptable acid addition salts and the stereochemically isomeric forms thereof, wherein

the dotted line represents an optional bond;

X is oxygen or sulfur;

-A- is a bivalent radical of formula

—CH═CH— (a-1),

—CH₂—CH₂— (a-2),

—CH₂—CH₂—CH₂— (a-3),

—CH₂—O— (a4),

—CH₂—CH₂—O— (a-5),

—CH₂—S— (a-6),

—CH₂—CH₂—S— (a-7),

—CH═N— (a-8),

—N═N— (a-9), or

—CO—NH— (a-10);

wherein optionally one hydrogen atom may be replaced by C₁₋₄alkyl or Ar¹;

R¹ and R² each independently are hydrogen, hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkyloxyC₁₋₆alkyloxy, C₁₋₆alkyloxycarbonyl, aminoC₁₋₆alkyloxy, mono- or di(C₁₋₆alkyl)aminoC₁₋₆alkyloxy, Ar², Ar²—C₁₋₆alkyl, Ar²-oxy, Ar²—C₁₋₆alkyloxy; or when on adjacent positions R¹ and R² taken together may form a bivalent radical of formula

—O—CH₂—O— (b-1),

—O—CH₂—CH₂—O— (b-2),

—O—CH═CH— (b-3),

—O—CH₂—CH₂— (b-4),

—O—CH₂—CH₂—CH₂— (b-5), or

—CH═CH—CH═CH— (b-6);

R³ and R⁴ each independently are hydrogen, halo, cyano, C₁₋₆alkyl, C₁₋₆alkyloxy, Ar³-oxy, C₁₋₆alkylthio, di(C₁₋₆alkyl)amino, trihalomethyl, trihalomethoxy, or

when on adjacent positions R³ and R⁴ taken together may form a bivalent radical of formula

—O—CH₂—O— (c-1),

—O—CH₂—CH₂—O— (c-2), or

—CH═CH—CH═CH— (c-3);

R⁵ is a radical of formula

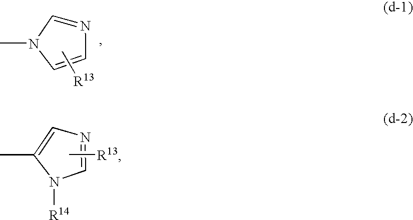

wherein R¹³ is hydrogen, halo, Ar⁴, C₁₋₆alkyl, hydroxyC₁₋₆alkyl, C₁₋₆alkyloxyC₁₋₆alkyl, C₁₋₆alkyloxy, C₁₋₆alkylthio, amino, C₁₋₆alkyloxycarbonyl, C₁₋₆alkylS(O)C₁₋₆alkyl or C₁₋₆alkylS(O)₂C₁₋₆alkyl;

R¹⁴ is hydrogen, C₁₋₆alkyl or di(C₁₋₄alkyl)aminosulfonyl;

R⁶ is hydrogen, hydroxy, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, C₁₋₆alkyloxycarbonylC₁₋₆alkyl, C₁₋₆alkylcarbonyl-C₁₋₆alkyl, C₁₋₆alkyloxycarbonyl, mono- or di(C₁₋₆alkyl)aminoC₁₋₆alkyl, Ar⁵, Ar⁵—C₁₋₆alkyloxyC₁₋₆alkyl; or a radical of formula

—O—R⁷ (e-1),

—S—R⁷ (e-2),

—N—R⁸R⁹ (e-3),

wherein R⁷ is hydrogen, C₁₋₆alkyl, C₁₋₆alkylcarbonyl, Ar⁶, Ar⁶—C₁₋₆alkyl, C₁₋₆alkyloxycarbonylC₁₋₆alkyl, or a radical of formula -Alk-OR¹⁰ or -Alk-NR¹¹R¹²;

R⁸ is hydrogen, C₁₋₆alkyl, Ar⁷ or Ar⁷—C₁₋₆alkyl;

R⁹ is hydrogen, C₁₋₆alkyl, C₁₋₆alkylcarbonyl, C₁₋₆alkyloxycarbonyl, C₁₋₆alkylaminocarbonyl, Ar⁸, Ar⁸—C₁₋₆alkyl, C₁₋₆alkylcarbonyl-C₁₋₆alkyl, Ar⁸-carbonyl, Ar⁸—C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical or formula -Alk-OR¹⁰ or -Alk-NR¹¹R¹²;

wherein Alk is C₁₋₆alkanediyl;

R¹⁰ is hydrogen, C₁₋₆alkyl, C₁₋₆alkylcarbonyl, hydroxyC₁₋₆alkyl, Ar⁹ or Ar⁹—C₁₋₆alkyl;

R¹¹ is hydrogen, C₁₋₆alkyl, C₁₋₆alkylcarbonyl, Ar¹⁰ or Ar¹⁰—C₁₋₆alkyl;

R¹² is hydrogen, C₁₋₆alkyl, Ar¹¹ or Ar¹¹—C₁₋₆alkyl; and

Ar¹ to Ar¹¹ are each independently selected from phenyl; or phenyl substituted with halo, C₁₋₆alkyl, C₁₋₆alkyloxy or trifluoromethyl.

WO-98/49157, concerns the preparation, formulation and pharmaceutical properties of farnesyl protein transferase inhibiting compounds of formula (VIII)

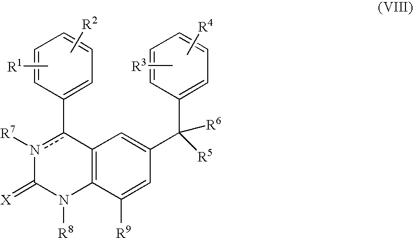

the pharmaceutically acceptable acid addition salts and the stereochemically isomeric forms thereof, wherein

the dotted line represents an optional bond;

X is oxygen or sulfur;

R¹ and R² each independently are hydrogen, hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkyloxyC₁₋₆alkyloxy, C₁₋₆alkyloxycarbonyl, aminoC₁₋₆alkyloxy, mono- or di(C₁₋₆alkyl)aminoC₁₋₆alkyloxy, Ar¹, Ar¹C₁₋₆alkyl, Ar¹ oxy or Ar¹C₁₋₆alkyloxy;

R³ and R⁴ each independently are hydrogen, halo, cyano, C₁₋₆alkyl, C₁₋₆alkyloxy, Ar¹oxy, C₁₋₆alkylthio, di(C₁₋₆alkyl)amino, trihalomethyl or trihalomethoxy;

R⁵ is hydrogen, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, C₁₋₆alkyloxycarbonylC₁₋₆alkyl, C₁₋₆alkylcarbonyl-C₁₋₆alkyl, C₁₋₆alkyloxycarbonyl, mono- or di(C₁₋₆alkyl)aminoC₁₋₆alkyl, Ar¹, Ar¹C₁₋₆alkyloxyC₁₋₆alkyl; or a radical of formula

—O—R¹⁰ (a-1),

—S—R¹⁰ (a-2),

—N—R¹¹R¹² (a-3),

wherein R¹⁰ is hydrogen, C₁₋₆alkyl, C₁₋₆alkylcarbonyl, Ar¹, Ar¹C₁₋₆alkyl, C₁₋₆alkyloxycarbonylC₁₋₆alkyl, or a radical of formula -Alk-OR¹³ or-Alk-NR¹⁴R¹⁵;

R¹¹ is hydrogen, C₁₋₆alkyl, Ar¹ or Ar¹C₁₋₆alkyl;

R¹² is hydrogen, C₁₋₆alkyl, C₁₋₆alkylcarbonyl, C₁₋₆alkyloxycarbonyl, C₁₋₆alkylaminocarbonyl, Ar¹, Ar¹C₁₋₆alkyl, C₁₋₆alkylcarbonyl-C₁₋₆alkyl, Ar¹carbonyl, Ar¹C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical or formula -Alk-OR¹³ or -Alk-NR¹⁴R¹⁵; wherein Alk is C₁₋₆alkanediyl;

R¹³ is hydrogen, C₁₋₆alkyl, C₁₋₆alkylcarbonyl, hydroxyC₁₋₆alkyl, Ar¹ or Ar¹C₁₋₆alkyl;

R¹⁴ is hydrogen, C₁₋₆alkyl, Ar¹ or Ar¹C₁₋₆alkyl;

R¹⁵ is hydrogen, C₁₋₆alkyl, C₁₋₆alkylcarbonyl, Ar¹ or Ar¹C₁₋₆alkyl;

R⁶ is a radical of formula

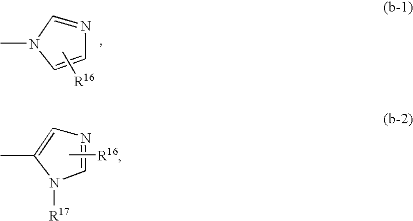

wherein R¹⁶ is hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, C₁₋₆alkyloxycarbonyl, C₁₋₆alkylthioC₁₋₆alkyl, C₁₋₆alkylS(O)C₁₋₆alkyl or C₁₋₆alkylS(O)₂C₁₋₆alkyl;

R¹⁷ is hydrogen, C₁₋₆alkyl or di(C₁₋₄alkyl)aminosulfonyl;

R⁷ is hydrogen or C₁₋₆alkyl provided that the dotted line does not represent a bond;

R⁸ is hydrogen, C₁₋₆alkyl or Ar²CH₂ or Het¹CH₂;

R⁹ is hydrogen, C₁₋₆alkyl, C₁₋₆alkyloxy or halo; or

R⁸ and R⁹ taken together to form a bivalent radical of formula

—CH═CH— (c-1),

—CH₂—CH₂— (c-2),

—CH₂—CH₂—CH₂— (c-3),

—CH₂—O— (c-4), or

—CH₂—CH₂—O— (c-5);

Ar¹ is phenyl; or phenyl substituted with 1 or 2 substituents each independently selected from halo, C₁₋₆alkyl, C₁₋₆alkyloxy or trifluoromethyl;

Ar² is phenyl; or phenyl substituted with 1 or 2 substituents each independently selected from halo, C₁₋₆alkyl, C₁₋₆alkyloxy or trifluoromethyl; and

Het¹ is pyridinyl; pyridinyl substituted with 1 or 2 substituents each independently selected from halo, C₁₋₆alkyl, C₁₋₆alkyloxy or trifluoromethyl.

As used in the foregoing definitions and hereinafter for compounds of formulae (I), (II), (III), (IV), (V), (VI), (VII) and (VIII) halo defines fluoro, chloro, bromo and iodo; C₁₋₆alkyl defines straight and branched chained saturated hydrocarbon radicals having from 1 to 6 carbon atoms such as, for example, methyl, ethyl, propyl, butyl, pentyl, hexyl and the like; C₁₋₈alkyl encompasses the straight and branched chained saturated hydrocarbon radicals as defined in C₁₋₆alkyl as well as the higher homologues thereof containing 7 or 8 carbon atoms such as, for example heptyl or octyl; C₁₋₁₂alkyl again encompasses C₁₋₈alkyl and the higher homologues thereof containing 9 to 12 carbon atoms, such as, for example, nonyl, decyl, undecyl, dodecyl; C₁₋₁₆alkyl again encompasses C₁₋₁₂alkyl and the higher homologues thereof containing 13 to 16 carbon atoms, such as, for example, tridecyl, tetradecyl, pentedecyl and hexadecyl; C₂₋₆alkenyl defines straight and branched chain hydrocarbon radicals containing one double bond and having from 2 to 6 carbon atoms such as, for example, ethenyl, 2-propenyl, 3-butenyl, 2-pentenyl, 3-pentenyl, 3-methyl-2-butenyl, and the like; C₁₋₆alkanediyl defines bivalent straight and branched chained saturated hydrocarbon radicals having from 1 to 6 carbon atoms, such as, for example, methylene, 1,2-ethanediyl, 1,3-propanediyl, 1,4-butanediyl, 1,5-pentanediyl, 1,6-hexanediyl and the branched isomers thereof. The term “C(═O)” refers to a carbonyl group, “S(O)” refers to a sulfoxide and “S(O)₂” to a sulfon. The term “natural amino acid” refers to a natural amino acid that is bound via a covalent amide linkage formed by loss of a molecule of water between the carboxyl group of the amino acid and the amino group of the remainder of the molecule. Examples of natural amino acids are glycine, alanine, valine, leucine, isoleucine, methionine, proline, phenylanaline, tryptophan, serine, threonine, cysteine, tyrosine, asparagine, glutamine, aspartic acid, glutamic acid, lysine, arginine, histidine.

WO-00/39082 concerns the preparation, formulation and pharmaceutical properties of farnesyl protein transferase inhibiting compounds of formula (IX)

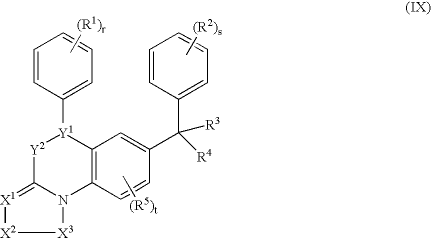

or the pharmaceutically acceptable acid addition salts and the stereochemically isomeric forms thereof, wherein

═X¹—X²—X³— is a trivalent radical of formula

═N—CR⁶═CR⁷— (x-1),

═N—N═CR⁶— (x-2),

═N—NH—C(═O)— (x-3),

═N—N═N— (x-4),

═N—CR⁶═N— (x-5),

═CR⁶—CR⁷═CR⁸— (x-6),

═CR⁶—N═CR⁷— (x-7),

═CR⁶—NH—C(═O)— (x-8), or

═CR⁶—N═N— (x-9);

wherein each R⁶, R⁷ and R⁸ are independently hydrogen, C₁₋₄alkyl, hydroxy, C₁₋₄alkyloxy, aryloxy, C₁₋₄alkyloxycarbonyl, hydroxyC₁₋₄alkyl, C₁₋₄alkyloxyC₁₋₄alkyl, mono- or di(C₁₋₄alkyl)aminoC₁₋₄alkyl, cyano, amino, thio, C₁₋₄alkylthio, arylthio or aryl;

>Y¹—Y²— is a trivalent radical of formula

>CH—CHR⁹— (y-1),

>C═N— (y-2),

>CH—NR⁹— (y-3), or

>C═CR⁹ (y-4);

wherein each R⁹ independently is hydrogen, halo, halocarbonyl, aminocarbonyl, hydroxyC₁₋₄alkyl, cyano, carboxyl, C₁₋₄alkyl, C₁₋₄alkyloxy, C₁₋₄alkyloxyC₁₋₄alkyl, C₁₋₄alkyloxycarbonyl, mono- or di(C₁₋₄alkyl)amino, mono- or di(C₁₋₄alkyl)aminoC₁₋₄alkyl, aryl;

r and s are each independently 0, 1, 2, 3, 4 or 5;

t is 0, 1, 2 or 3;

each R¹ and R² are independently hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkylthio, C₁₋₆alkyloxyC₁₋₆alkyloxy, C₁₋₆alkyloxycarbonyl, aminoC₁₋₆alkyloxy, mono- or di(C₁₋₆alkyl)amino, mono- or di(C₁₋₆alkyl)aminoC₁₋₆alkyloxy, aryl, arylC₁₋₆alkyl, aryloxy or arylC₁₋₆alkyloxy, hydroxycarbonyl, C₁₋₆alkyloxycarbonyl, aminocarbonyl, aminoC₁₋₆alkyl, mono- or di(C₁₋₆alkyl)aminocarbonyl, mono- or di(C₁₋₆alkyl)aminoC₁₋₆alkyl; or

two R¹ or R² substituents adjacent to one another on the phenyl ring may independently form together a bivalent radical of formula

—O—CH₂—O— (a-1),

—O—CH₂—CH₂—O— (a-2),

—O═CH═CH— (a-3),

—O—CH₂—CH₂— (a-4),

—O—CH₂—CH₂—CH₂— (a-5), or

—CH═CH—CH═CH— (a-6);

R³ is hydrogen, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, hydroxycarbonyl, hydroxycarbonylC₁₋₆alkyl, C₁₋₆alkyloxycarbonylC₁₋₆alkyl, C₁₋₆alkylcarbonylC₁₋₆alkyl, C₁₋₆alkyloxycarbonyl, aryl, arylC₁₋₆alkyloxyC₁₋₆alkyl, mono- or di(C₁₋₆alkyl)aminoC₁₋₆alkyl;

or a radical of formula

—O—R¹⁰ (b-1)

—S—R¹⁰ (b-2),

—NR¹¹R¹² (b-3),

wherein R¹⁰ is hydrogen, C₁₋₆alkyl, C₁₋₆alkylcarbonyl, aryl, arylC₁₋₆alkyl, C₁₋₆alkyloxycarbonylC₁₋₆alkyl, or a radical of formula -Alk-OR¹³ or -Alk-NR¹⁴R¹⁵;

R¹¹ is hydrogen, C₁₋₆alkyl, aryl or arylC₁₋₆alkyl;

R¹² is hydrogen, C₁₋₆alkyl, aryl, hydroxy, amino, C₁₋₆alkyloxy, C₁₋₆alkylcarbonylC₁₋₆alkyl, arylC₁₋₆alkyl, C₁₋₆alkylcarbonylamino, mono- or di(C₁₋₆alkyl)amino, C₁₋₆alkylcarbonyl, aminocarbonyl, arylcarbonyl, haloC₁₋₆alkylcarbonyl, arylC₁₋₆alkylcarbonyl, C₁₋₆alkyloxycarbonyl, C₁₋₆alkyloxyC₁₋₆alkylcarbonyl, mono- or di(C₁₋₆alkyl)aminocarbonyl wherein the alkyl moiety may optionally be substituted by one or more substituents independently selected from aryl or C₁₋₃alkyloxycarbonyl, aminocarbonylcarbonyl, mono- or di(C₁₋₆alkyl)aminoC₁₋₆alkylcarbonyl, or a radical or formula -Alk-OR¹³ or -Alk-NR¹⁴R¹⁵;

wherein Alk is C₁₋₆alkanediyl;

R¹³ is hydrogen, C₁₋₆alkyl, C₁₋₆alkylcarbonyl, hydroxyC₁₋₆alkyl, aryl or arylC₁₋₆alkyl;

R¹⁴ is hydrogen, C₁₋₆alkyl, aryl or arylC₁₋₆alkyl;

R¹⁵ is hydrogen, C₁₋₆alkyl, C₁₋₆alkylcarbonyl, aryl or arylC₁₋₆alkyl;

R⁴ is a radical of formula

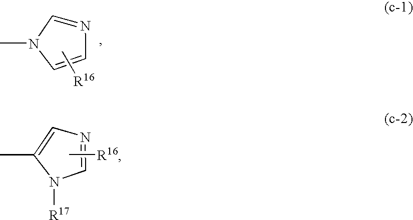

wherein R¹⁶ is hydrogen, halo, aryl, C₁₋₆alkyl, hydroxyC₁₋₆alkyl, C₁₋₆alkyloxyC₁₋₆alkyl, C₁₋₆alkyloxy, C₁₋₆alkylthio, amino, mono- or di(C₁₋₄alkyl)amino, hydroxycarbonyl, C₁₋₆alkyloxycarbonyl, C₁₋₆alkylthioC₁₋₆alkyl, C₁₋₆alkylS(O)C₁₋₆alkyl or C₁₋₆alkylS(O)₂C₁₋₆alkyl;

R¹⁶ may also be bound to one of the nitrogen atoms in the imidazole ring of formula (c-1) or (c-2), in which case the meaning of R¹⁶ when bound to the nitrogen is limited to hydrogen, aryl, C₁₋₆alkyl, hydroxyC₁₋₆alkyl, C₁₋₆alkyloxyC₁₋₆alkyl, C₁₋₆alkyloxycarbonyl, C₁₋₆alkylS(O)C₁₋₆alkyl or C₁₋₆alkylS(O)₂C₁₋₆alkyl;

R¹⁷ is hydrogen, C₁₋₆alkyl, C₁₋₆alkyloxyC₁₋₆alkyl, arylC₁₋₆alkyl, trifluoromethyl or di(C₁₋₄alkyl)aminosulfonyl;

R⁵ is C₁₋₆alkyl, C₁₋₆alkyloxy or halo;

aryl is phenyl, naphthalenyl or phenyl substituted with 1 or more substituents each independently selected from halo, C₁₋₆alkyl, C₁₋₆alkyloxy or trifluoromethyl.

As used in the foregoing definitions and hereinafter for compounds of formula (IX), halo is generic to fluoro, chloro, bromo and iodo; C₁₋₄alkyl defines straight and branched chain saturated hydrocarbon radicals having from 1 to 4 carbon atoms such as, e.g. methyl, ethyl, propyl, butyl, 1-methylethyl, 2-methylpropyl and the like; C₁₋₆alkyl includes C₁₋₄alkyl and the higher homologues thereof having 5 to 6 carbon atoms such as, for example, pentyl, 2-methyl-butyl, hexyl, 2-methylpentyl and the like; C₁₋₆alkanediyl defines bivalent straight and branched chained saturated hydrocarbon radicals having from 1 to 6 carbon atoms, such as, for example, methylene, 1,2-ethanediyl, 1,3-propanediyl, 1,4-butanediyl, 1,5-pentanediyl, 1,6-hexanediyl and the branched isomers thereof; C₂₋₆alkenyl defines straight and branched chain hydrocarbon radicals containing one double bond and having from 2 to 6 carbon atoms such as, for example, ethenyl, 2-propenyl, 3-butenyl, 2-pentenyl, 3-pentenyl, 3-methyl-2-butenyl, and the like. The term “S(O)” refers to a sulfoxide and “S(O)₂” to a sulfon.

Unexpectedly, we have now found that the farnesyl protein transferase inhibitors identified supra, which may hereinafter be referred to as compounds according to the present invention, are useful for the treatment of IBD, including but not limited to Crohn's and ulcerative colitis.

Accordingly, the present invention relates to the use of a farnesyl protein transferase inhibitor selected from compounds of formulae (I), (II), (Ill), (IV), (V), (VI), (VII), (VIII) and (IX) in the manufacture of a medicament for the treatment of inflammatory bowel disease, for example, Crohn's and ulcerative colitis.

The treatment of inflammatory bowel diseases includes both the treatment of the acute disease state, thereby inducing remission of the disease or improvement of the lesions or clinical condition, as well as the use in maintenance therapy.

The invention further includes a method of treatment of inflammatory bowel disease in a mammal, including a human, by administering a therapeutically effective amount of a compound according to the present invention.

This invention further includes a method for preventing inflammatory bowel disease in a mammal, including a human, by administering a therapeutically effective amount of a compound according to the present invention. In particular, the present invention is concerned with the use of a farnesyl protein transferase inhibitor for the preparation of a pharmaceutical composition for treating inflammatory bowel disease, wherein said farnesyl protein transferase inhibitor is an (imidazoly-5-yl)methyl-2-quinolinone derivative of formula (I), or a compound of formula (II) or (III) which is metabolized in vivo to the compound of formula (I), said compounds being represented by

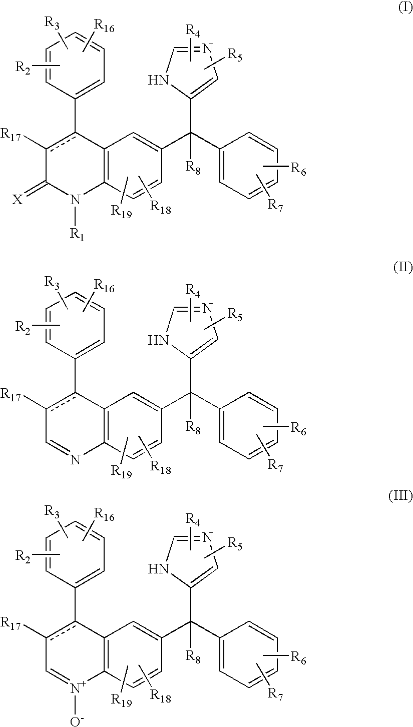

the pharmaceutically acceptable acid or base addition salts and the stereochemically isomeric forms thereof, wherein

the dotted line represents an optional bond;

X is oxygen or sulfur;

R¹ is hydrogen, C₁₋₁₂alkyl, Ar¹, Ar²C₁₋₆alkyl, quinolinylC₁₋₆alkyl, pyridylC₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl, or a radical of formula -Alk¹-C(═O)—R⁹, -Alk¹-S(O)—R⁹ or -Alk¹-S(O)₂—R⁹, whereinAlk¹ is C₁₋₆alkanediyl,

R⁹ is hydroxy, C₁₋₆alkyl, C₁₋₆alkyloxy, amino, C₁₋₈alkylamino or C₁₋₈alkylamino substituted with C₁₋₆alkyloxycarbonyl;

R², R³ and R¹⁶ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, aminoC₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl, 4,4-dimethyloxazolyl; or

when on adjacent positions R² and R³ taken together may form a bivalent radical of formula

—O—CH₂—O— (a-1),

—O—CH₂—CH₂—O— (a-2),

—O—CH═CH— (a-3),

—O—CH₂—CH₂— (a-4),

—O—CH₂—CH₂—CH₂— (a-5), or

—CH═CH—CH═CH— (a-6);

R⁴ and R⁵ each independently are hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;

R⁶ and R⁷ each independently are hydrogen, halo, cyano, C₁₋₆alkyl, C₁₋₆alkyloxy, Ar²oxy, trihalomethyl, C₁₋₆alkylthio, di(C₁₋₆alkyl)amino, or when on adjacent positions R⁶ and R⁷ taken together may form a bivalent radical of formula

—O—CH₂—O— (c-1), or

—CH═CH—CH═CH— (c-2);

R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, carboxyC₁₋₆alkyl, hydroxyC₁₋₆alkyl, aminoC₁₋₆alkyl, mono- or di(C₁₋₆alkyl)aminoC₁₋₆alkyl, imidazolyl, haloC₁₋₆alkyl, C₁₋₆alkyloxyC₁₋₆alkyl, aminocarbonylC₁₋₆alkyl, or a radical of formula

—O—R¹⁰ (b-1),

—S—R¹⁰ (b-2),

—N—R¹¹R¹² (b-3),

wherein R¹⁰ is hydrogen, C₁₋₆alkyl, C₁₋₆alkylcarbonyl, Ar¹, Ar²C₁₋₆alkyl, C₁₋₆alkyloxycarbonylC₁₋₆alkyl, or a radical or formula -Alk²-OR¹³ or -Alk²-NR¹⁴R¹⁵;

R¹¹ is hydrogen, C₁₋₁₂alkyl, Ar¹ or Ar²C₁₋₆alkyl;

R¹² is hydrogen, C₁₋₆alkyl, C₁₋₁₆alkylcarbonyl, C₁₋₆alkyloxycarbonyl, C₁₋₆alkylaminocarbonyl, Ar¹, Ar²C₁₋₆alkyl, C₁₋₆alkylcarbonylC₁₋₆alkyl, a natural amino acid, Ar¹ carbonyl, Ar²C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical or formula -Alk²-OR¹³ or -Alk²-NR¹⁴R¹⁵;

wherein Alk² is C₁₋₆alkanediyl;

R¹³ is hydrogen, C₁₋₆alkyl, C₁₋₆alkylcarbonyl, hydroxyC₁₋₆alkyl, Ar¹ or Ar²C₁₋₆alkyl;

R¹⁴ is hydrogen, C₁₋₆alkyl, Ar¹ or Ar²C₁₋₆alkyl;

R¹⁵ is hydrogen, C₁₋₆alkyl, C₁₋₆alkylcarbonyl, Ar¹ or Ar²C₁₋₆alkyl;

R¹⁷ is hydrogen, halo, cyano, C₁₋₆alkyl, C₁₋₆alkyloxycarbonyl, Ar¹;

R¹⁸ is hydrogen, C₁₋₆alkyl, C₁₋₆alkyloxy or halo;

R¹⁹ is hydrogen or C₁₋₆alkyl;

Ar¹ is phenyl or phenyl substituted with C₁₋₆alkyl, hydroxy, amino, C₁₋₆alkyloxy or halo; and

Ar² is phenyl or phenyl substituted with C₁₋₆alkyl, hydroxy, amino, C₁₋₆alkyloxy or halo.

In Formulae (I), (II) and (III), R⁴ or R⁵ may also be bound to one of the nitrogen atoms in the imidazole ring. In that case the hydrogen on the nitrogen is replaced by R⁴ or R⁵ and the meaning of R⁴ and R⁵ when bound to the nitrogen is limited to hydrogen, Ar¹, C₁₋₆alkyl, hydroxyC₁₋₆alkyl, C₁₋₆alkyloxyC₁₋₆alkyl, C₁₋₆alkyloxycarbonyl, C₁₋₆alkylS(O)C₁₋₆alkyl, C₁₋₆alkylS(O)₂C₁₋₆alkyl.

Preferably the substituent R¹⁸ is situated on the 5 or 7 position of the quinolinone moiety and substituent R¹⁹ is situated on the 8 position when R¹⁸ is on the 7-position.

Interesting compounds are these compounds of formula (I) wherein X is oxygen.

Also interesting compounds are these compounds of formula (I) wherein the dotted line represents a bond, so as to form a double bond.

Another group of interesting compounds are those compounds of formula (I) wherein R¹ is hydrogen, C₁₋₆alkyl, C₁₋₆alkyloxyC₁₋₆alkyl, di(C₁₋₆alkyl)aminoC₁₋₆alkyl, or a radical of formula -Alk¹-C(═O)—R⁹, wherein Alk¹ is methylene and R⁹ is C₁₋₈alkylamino substituted with C₁₋₆alkyloxycarbonyl.

Still another group of interesting compounds are those compounds of formula (I) wherein R³ is hydrogen or halo; and R² is halo, C₁₋₆alkyl, C₂₋₆alkenyl, C₁₋₆alkyloxy, trihalomethoxy or hydroxyC₁₋₆alkyloxy.

A further group of interesting compounds are those compounds of formula (I) wherein R² and R³ are on adjacent positions and taken together to form a bivalent radical of formula (a-1), (a-2) or (a-3).

A still further group of interesting compounds are those compounds of formula (I) wherein R⁵ is hydrogen and R⁴ is hydrogen or C₁₋₆alkyl.

Yet another group of interesting compounds are those compounds of formula (I) wherein R⁷ is hydrogen; and R⁶ is C₁₋₆alkyl or halo, preferably chloro, especially 4-chloro.

A particular group of compounds are those compounds of formula (I) wherein R⁸ is hydrogen, hydroxy, haloC₁₋₆alkyl, hydroxyC₁₋₆alkyl, cyanoC₁₋₆alkyl, C₁₋₆alkyloxycarbonylC₁₋₆alkyl, imidazolyl, or a radical of formula —NR¹¹R¹² wherein R¹¹ is hydrogen or C₁₋₁₂alkyl and R¹² is hydrogen, C₁₋₆alkyl, C₁₋₆alkyloxy, hydroxy, C₁₋₆alkyloxyC₁₋₆alkylcarbonyl, or a radical of formula -Alk²-OR¹³ wherein R¹³ is hydrogen or C₁₋₆alkyl.

Preferred compounds are those compounds wherein R¹ is hydrogen, C₁₋₆alkyl, C₁₋₆alkyloxyC₁₋₆alkyl, di(C₁₋₆alkyl)aminoC₁₋₆alkyl, or a radical of formula -Alk¹-C(═O)—R⁹, wherein Alk¹ is methylene and R⁹ is C₁₋₈alkylamino substituted with C₁₋₆alkyloxycarbonyl; R² is halo, C₁₋₆alkyl, C₂₋₆alkenyl, C₁₋₆alkyloxy, trihalomethoxy, hydroxyC₁₋₆alkyloxy or Ar¹; R³ is hydrogen; R⁴ is methyl bound to the nitrogen in 3-position of the imidazole; R⁵ is hydrogen; R⁶ is chloro; R⁷ is hydrogen; R⁸ is hydrogen, hydroxy, haloC₁₋₆alkyl, hydroxyC₁₋₆alkyl, cyanoC₁₋₆alkyl, C₁₋₆alkyloxycarbonylC₁₋₆alkyl, imidazolyl, or a radical of formula —NR¹¹ R¹² wherein R¹¹ is hydrogen or C₁₋₁₂alkyl and R¹² is hydrogen, C₁₋₆alkyl, C₁₋₆alkyloxy, C₁₋₆alkyloxyC₁₋₆alkylcarbonyl, or a radical of formula -Alk²-OR¹³ wherein R¹³ is C₁₋₆alkyl; R¹⁷ is hydrogen and R¹⁸ is hydrogen.

Most preferred compounds are

4-(3-chlorophenyl)-6-[(4-chlorophenyl)hydroxy(1-methyl-1H-imidazol-5-yl)methyl]-1-methyl-2(1H)-quinolinone,

6-[amino(4-chlorophenyl)-1-methyl-1H-imidazol-5-ylmethyl]-4-(3-chlorophenyl)-1-methyl-2(1H)-quinolinone;

6-[(4-chlorophenyl)hydroxy(1-methyl-1H-imidazol-5-yl)methyl]-4-(3-ethoxyphenyl)-1-methyl-2(1H)-quinolinone;

6-[(4-chlorophenyl)(1-methyl-1H-imidazol-5-yl)methyl]-4-(3-ethoxyphenyl)-1-methyl-2(1H)-quinolinone monohydrochloride monohydrate;

6-[amino(4-chlorophenyl)(1-methyl-1H-imidazol-5-yl)methyl]-4-(3-ethoxyphenyl)-1-methyl-2(1H)-quinolinone,

6-amino(4-chlorophenyl)(1-methyl-1H-imidazol-5-yl)methyl]-1-methyl-4-(3propylphenyl)-2(1H)-quinolinone; a stereoisomeric form thereof or a pharmaceutically acceptable acid or base addition salt; and

(+)-6-[amino(4-chlorophenyl)(1-methyl-1H-imidazol-5-yl)methyl]-4-(3-chlorophenyl)-1-methyl-2(1H)-quinolinone (Compound 75 in Table 1 of the Experimental part of WO 97/21701); or a pharmaceutically acceptable acid addition salt thereof.

Further preferred embodiments of the present invention include compounds of formula (IX) wherein one or more of the following restrictions apply:

═X¹—X²—X³ is a trivalent radical of formula (x-1), (x-2), (x-3), (x-4) or (x-9) wherein each R⁶ independently is hydrogen, C₁₋₄-alkyl, C₁₋₄alkyloxycarbonyl, amino or aryl and R⁷ is hydrogen;

>Y¹—Y²— is a trivalent radical of formula (y-1), (y-2), (y-3), or (y-4) wherein each R⁹ independently is hydrogen, halo, carboxyl, C₁₋₄alkyl or C₁₋₄alkyloxycarbonyl;

r is 0, 1 or 2;

s is 0 or 1;

t is 0;

R¹ is halo, C₁₋₆alkyl or two R¹ substituents ortho to one another on the phenyl ring may independently form together a bivalent radical of formula (a-1);

R² is halo;

R³ is halo or a radical of formula (b-1) or (b-3) wherein

R¹⁰ is hydrogen or a radical of formula -Alk-OR¹³.

R¹¹ is hydrogen;

R¹² is hydrogen, C₁₋₆alkyl, C₁₋₆alkylcarbonyl, hydroxy, C₁₋₆alkyloxy or mono- or

di(C₁₋₆alkyl)aminoC₁₋₆alkylcarbonyl;

Alk is C₁₋₆alkanediyl and R¹³ is hydrogen;

R⁴ is a radical of formula (c-1) or (c-2) wherein

R¹⁶ is hydrogen, halo or mono- or di(C₁₋₄alkyl)amino;

R¹⁷ is hydrogen or C₁₋₆alkyl;

aryl is phenyl.

A particular group of compounds consists of those compounds of formula (IX) wherein

═X¹—X²—X³ is a trivalent radical of formula (x-1), (x-2), (x-3) or (x-9), >Y1—Y2 is a trivalent radical of formula (y-2), (y-3) or (y-4), r is 0 or 1, s is 1, t is 0, R¹ is halo, C₍₁₋₄₎alkyl or forms a bivalent radical of formula (a-1), R² is halo or C₁₋₄alkyl, R³ is hydrogen or a radical of formula (b-1) or (b-3), R⁴ is a radical of formula (c-1) or (c-2), R⁶ is hydrogen, C₁₋₄alkyl or phenyl, R⁷ is hydrogen, R⁹ is hydrogen or C₁₋₄alkyl, R¹⁰ is hydrogen or -Alk-OR¹³, R¹¹ is hydrogen and R¹² is hydrogen or C₁₋₆alkylcarbonyl and R¹³ is hydrogen;

Preferred compounds are those compounds of formula (IX) wherein ═X¹—X²—X³ is a trivalent radical of formula (x-1), >Y1—Y2 is a trivalent radical of formula (y-4), r is 0 or 1, s is 1, t is 0, R¹ is halo, preferably chloro and most preferably 3-chloro, R² is halo, preferably 4-chloro or 4-fluoro, R³ is hydrogen or a radical of formula (b-1) or (b-3), R⁴ is a radical of formula (c-1) or (c-2), R⁶ is hydrogen, R⁷ is hydrogen, R⁹ is hydrogen, R¹⁰ is hydrogen, R¹¹ is hydrogen and R¹² is hydrogen;

Other preferred compounds are those compounds of formula (IX) wherein ═X¹—X²—X³ is a trivalent radical of formula (x-2) or (x-3), >Y1—Y2 is a trivalent radical of formula (y-2), (y-3) or (y-4), r and s are 1, t is 0, R¹ is halo, preferably chloro, and most preferably 3-chloro or R¹ is C₁₋₄alkyl, preferably 3-methyl, R² is halo, preferably chloro, and most preferably 4-chloro, R³ is a radical of formula (b-1) or (b-3), R⁴ is a radical of formula (c-2), R⁶ is C₁₋₄alkyl, R⁹ is hydrogen, R¹⁰ and R¹¹ are hydrogen and R¹² is hydrogen or hydroxy;

The most preferred compounds of formula (IX) are

7-[(4-fluorophenyl)(1H-imidazol-1-yl)methyl]-5-phenylimidazo[1,2-a]quinoline;

α-(4-chlorophenyl)-α-(1-methyl-1H-imidazol-5-yl)-5-phenylimidazo[1,2-a]quinoline-7-methanol;

5-(3-chlorophenyl)-α-(4-chlorophenyl)-α-(1-methyl-1H-imidazol-5-yl)-imidazo[1,2-a]quinoline-7-methanol;

5-(3-chlorophenyl)-α-(4-chlorophenyl)-α-(1-methyl-1H-imidazol-5-yl)imidazo[1,2-a]quinoline-7-methanamine;

5-(3-chlorophenyl)-α-(4-chlorophenyl)-α-(1-methyl-1H-imidazol-5-yl)tetrazolo[1,5-a]quinoline-7-methanamine;

5-(3-chlorophenyl)-α-(4-chlorophenyl)-1-methyl-α-(1-methyl-1H-imidazol-5-yl)-1,2,4-triazolo[4,3-a]quinoline-7-methanol;

5-(3-chlorophenyl)-α-(4-chlorophenyl)-α-(1-methyl-1H-imidazol-5-yl)tetrazolo[1,5-a]quinoline-7-methanamine;

5-(3-chlorophenyl)-α-(4-chlorophenyl)-α-(1-methyl-1H-imidazol-5-yl)tetrazolo[1,5-a]quinazoline-7-methanol; 5-(3-chlorophenyl)-α-(4-chlorophenyl)-4,5-dihydro-α-(1-methyl-1H-imidazol-5-yl)tetrazolo[1,5-a]quinazoline-7-methanol;

5-(3-chlorophenyl)-α-(4-chlorophenyl)-α-(1-methyl-1H-imidazol-5-yl)tetrazolo[1,5-a]quinazoline-7-methanamine;

5-(3-chlorophenyl)-α-(4-chlorophenyl)-N-hydroxy-α-(1-methyl-1H-imidazol-5-yl)tetrahydro[1,5-a]quinoline-7-methanamine;

α-(4-chlorophenyl)-α-(1-methyl-1H-imidazol-5-yl)-5-(3-methylphenyl)tetrazolo[1,5-a]quinoline-7-methanamine; the pharmaceutically acceptable acid addition salts and the stereochemically isomeric forms thereof.

The pharmaceutically acceptable acid or base addition salts as mentioned hereinabove are meant to comprise the therapeutically active non-toxic acid and non-toxic base addition salt forms which the compounds of formulae (I), (II), (III), (IV), (V), (VI), (VII), (VIII) and (IX) are able to form. The compounds of formulae (I), (II), (III), (IV), (V), (VI), (VII), (VIII) and (IX) which have basic properties can be converted in their pharmaceutically acceptable acid addition salts by treating said base form with an appropriate acid. Appropriate acids comprise, for example, inorganic acids such as hydrohalic acids, e.g. hydrochloric or hydrobromic acid; sulfuric; nitric; phosphoric and the like acids; or organic acids such as, for example, acetic, propanoic, hydroxyacetic, lactic, pyruvic, oxalic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The compounds of formulae (I), (II), (III), (IV), (V), (VI), (VII), (VIII) and (IX) which have acidic properties may be converted in their pharmaceutically acceptable base addition salts by treating said acid form with a suitable organic or inorganic base. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The terms acid or base addition salt also comprise the hydrates and the solvent addition forms which the compounds of formulae (I), (II), (III), (IV), (V), (VI), (VII), (VIII) and (IX) are able to form. Examples of such forms are e.g. hydrates, alcoholates and the like.

The term stereochemically isomeric forms of compounds of formulae (I), (II), (III), (IV), (V), (VI), (VII), (VIII) and (IX), as used hereinbefore, defines all possible compounds made up of the same atoms bonded by the same sequence of bonds but having different three-dimensional structures which are not interchangeable, which the compounds of formulae (I), (II), (III), (IV), (V), (VI), (VII), (VIII) and (IX) may possess. Unless otherwise mentioned or indicated, the chemical designation of a compound encompasses the mixture of all possible stereochemically isomeric forms which said compound may possess. Said mixture may contain all diastereomers and/or enantiomers of the basic molecular structure of said compound. All stereochemically isomeric forms of the compounds of formulae (I), (II), (III), (IV), (V), (VI), (VII), (VIII) and (IX) both in pure form or in admixture with each other are intended to be embraced within the scope of the present invention.

Some of the compounds of formulae (I), (II), (III), (IV), (V), (VI), (VII), (VIII) and (IX) may also exist in their tautomeric forms. Such forms although not explicitly indicated in the above formula are intended to be included within the scope of the present invention.

Whenever used hereinafter, the term “compounds of formulae (I), (II), (III), (IV), (V), (VI), (VII), (VIII) and (IX)” is meant to include also the pharmaceutically acceptable acid or base addition salts and all stereoisomeric forms.

Farnesyl protein transferase inhibitors can be prepared and formulated into pharmaceutical compositions by methods known in the art and in particular according to the methods described in the published patent specifications mentioned herein and incorporated by reference; for the compounds of formulae (I), (II) and (III) suitable examples can be found in WO-97/21701. Compounds of formulae (IV), (V), and (VI) can be prepared and formulated using methods described in WO 97/16443, compounds of formulae (VII) and (VIII) according to methods described in WO 98/40383 and WO 98/49157 and compounds of formula (IX) according to methods described in WO 00/39082 respectively. To prepare the aforementioned medicaments, a therapeutically effective amount of the particular compound, optionally in addition salt form, as the active ingredient is combined in intimate admixture with a pharmaceutically acceptable carrier, which may take a wide variety of forms depending on the form of preparation desired for administration. The carrier(s) must be acceptable in the sense of being compatible with the other ingredients of the formula and not deleterious to the recipient thereof.

These pharmaceutical compositions are desirably in unitary dosage form suitable, preferably, for systemic administration such as oral, rectal, percutaneous, or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed.

For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable solutions containing compounds of formula (I) may be formulated in an oil for prolonged action. Appropriate oils for this purpose are, for example, peanut oil, sesame oil, cottonseed oil, corn oil, soy bean oil, synthetic glycerol esters of long chain fatty acids and mixtures of these and other oils. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment. As appropriate compositions for topical application there may be cited all compositions usually employed for topically administering drugs e.g. creams, gellies, dressings, shampoos, tinctures, pastes, ointments, salves, powders and the like. Application of said compositions may be by aerosol, e.g. with a propellent such as nitrogen, carbon dioxide, a freon, or without a propellent such as a pump spray, drops, lotions, or a semisolid such as a thickened composition which can be applied by a swab. In particular, semisolid compositions such as salves, creams, gellies, ointments and the like will conveniently be used.

For rectal administration, the pharmaceutical compositions may be presented as suppositories or as enemas. For rectal administration wherein the carrier is a solid, unit dose suppositories are preferred. Suitable carriers include cocoa butter and other materials commonly used in the art.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

Preferably, a therapeutic effective amount of the medicament comprising a compound according to the present invention is administered orally or parenterally. Said therapeutically effective amount is the amount that is effective in treating or preventing inflammatory bowel disease. The amount of compound according to the present invention, which is required to achieve a therapeutic effect will, of course, vary with the particular compound, route of administration, the age and condition of the recipient, and the particular disorder being treated.

On the basis of the current data, it appears that a pharmaceutical composition comprising a compound of formulae (I), (II), (III), (IV), (V), (VI), (VII), or (VIII) and in particular (+)-6-[amino(4-chlorophenyl) (1-methyl-1H-imidazol-5-yl)methyl]-4-(3-chlorophenyl)-1-methyl-2(1H)-quinolinone (compound 75) as the active ingredient can be administered orally in an amount of from 10 to 1500 mg daily, either as a single dose or subdivided into more than one dose. A preferred amount ranges from 100 to 1,000 mg daily, including 50 to 1,000 mg daily. A particularly preferred dosage for such a compound is 300 mg administered twice daily. This treatment can be given either continuously or intermittently in cycles of 3-4 weeks with treatment given for 1-21 days per cycle.

The compounds according to the present invention may also be used in accordance with the present invention in combination with one or more IBD treatment agents, including agents known to have utility for alleviating the symptoms of inflammatory bowel disease, including but not limited to:

1. Anti-inflammatory agents, e.g. sulfasalazine, olsalazine

2. Agents that control intestinal tract infection e.g. antibiotics:

(i) penicillins, e.g. bacitracin

(ii) glycopeptides, e.g. vancomycin

3. Steroids corticosteroids, e.g. prednisolone budenoside

4. Immuno-modulating agents, e.g. azathioprine, 6-MP

5. Agents for controlling diarrhea, e.g; cholestryramine

The various components of the combination described herein may be administered simultaneously (e.g. in separate or unitary compositions) or sequentually in either order and optionally via different routes.

Testing Method for Inflammatory Bowel Disease

The purpose of this study was to identify, characterize and document the preventative actions of compound 75 above, namely (+)-6-[amino(4-chlorophenyl) (1-methyl-1H-imidazol-5-yl)methyl]-4-(3-chlorophenyl)-1-methyl-2(1H)-quinolinone, a compound of formulae (I), in an experimental model of inflammatory bowel disease in mice.

Procedure

A group of female Swiss-Webster mice was received from Ace Animals, Inc., Boyertown, Pa. The animals were grouped housed (5 mice/cage) in plastic cages with water absorbent bedding. The animal room was temperature controlled and had a 12-hour light/dark cycle. The animals were fed Purina Rodent Chow #5012 and filtered tap water was supplied ad libitum by automatic watering system (during acclimation).

Following an acclimation period of 9 days, 40 healthy mice were selected for this study. The test animals were distributed (10 mice/group) into each one of the 4 test groups described below. There were no statistical significant differences between the mean group body weights.

| GROUP NUMBER | PRODUCT ID | DOSE LEVEL (mg/kg) |
|---|---|---|
| 1 | Vehicle no. DSS | 0 |
| 2 | Vehicle with DSS | 0 |
| 3 | Compound 75 with DSS | 25 PO bid |
| 4 | Compound 75 with DSS | 50 PO bid |

PO-oral
bid-twice per day (4 to 6 hours apart) days 0 through 6

Individual doses were calculated based on daily body weights. Prior to administration, Compound 75 was mixed with the vehicle (Beta cyclodextrin (40%) in 0.1 N HCL). This mixture was further diluted with 0.1 N HCL. The resulting mixture was then used to prepare the dilutions for groups 3 and 4. A 20% solution of Beta cyclodextrin in 0.1 N HCL was used for Groups 1 and 2 and constituted the vehicle control. Groups 2 through 4 were given a 5% w/w solution of dextran sulfate sodium (DSS) in distilled water substituted for their normal water supply (approximately 10 ml/mouse/day) from Days 0 through 6. Each group of animals received the appropriate amount of the test substance, as described above, by intubation using a stainless steel ball-tipped gavage needle attached to an appropriate syringe.

All mice were weighed daily and observed for signs of gross toxicity and behavioural changes, consistency of stool and presence of gross blood during the study. On Day 7, all animals were euthanized by CO₂ inhalation and necropsied. Following euthanasia, a stool sample was obtained from the colon of each animal and tested for occult blood (Quik-Cult, Laboratory Diagnostics Co., Morganville, N.J.). The colons were then removed and the length from the colo-cecal junction to the end of the distal rectum was measured. A segment of colon was collected from each animal and preserved in 10% formalin.

For each group, the disease activity index (DAI) was determined by evaluating changes in weight, Hemoccult positivity or gross bleeding and stool consistency using the following system.

Criteria for Scoring Disease Activity Index *

| Score | Weight Loss (%) | Stool # | Blood in feces |
|---|---|---|---|
| 0 | 0 or gain | Normal | Negative |
| 1 | 1-4.9 | — | +/− |
| 2 | 5.0-9.9 | Semi-solid | + |
| 3 | 10.15 | Diarrhoea | ++ |
| 4 | >15 | Bloody diarrhoea | Groos blood |

* DAI = (combined score of weight loss, stool consistency and bleeding)/3
# Normal stools = well-formed pellets; semi-solid = pasty stool that does not stick to the anus; diarrhoea = liquid stools that stick to the anus.

Data was analyzed using analysis of variance (single factor). Statistically methods included the Mann-Whitney test, ANOVA and Turkey-Kramer Multiple Comparisons test.

Statistical significance between test and control groups was established at a probability of p<0.05.

Results and Conclusion

Compound 75 showed significant activity in prevention of colitis produced by 5% Dextran Sulfate Sodium. Dose levels of 25 and 50 mg/kg PO bid prevented the shortening of the colon produced by DSS. The lower dose may have been more effective than the 50 mg/kg dose. Similar results were observed with the DAI (disease activity index) with or without the weight parameter. No significant protection against weight loss was noted with any dose. However, control mice receiving cyclodextrin lost weight over the duration of the study. In most studies vehicle treated mice usually gain weight. The weight loss observed with cyclodextrin+DSS was greater than that normally observed. Due to the severity of the weight loss in the control DSS group the study was terminated one day early. Eight deaths were noted in the study, 2 in the vehicle+DSS group, 1 each at the 25 and 50 mg/kg dose.

TABLE 1
EFFECT OF Compound 75 AFTER PO ADMINISTRATION BID:
PREVENTION OF DSS-INDUCED COLITIS IN MICE

| | Mean | % of | | | Blood |
|---|---|---|---|---|---|
| | Colon | Normal | DAI | DAINWT | in |
| Treatment (mg/kg) + | Length | Length | Mean ± | Mean ± | Feces |
| DSS | Cm ± SEM | Shortening | SEM | SEM | (%) |
| 1 Veh. PO no DSS | 11.8 ± 0.1 | 100 | 0.43 ± 0.11 | 0.20 ± 0.13 | 0 |
| 2 Veh. PO | 8.4 ± 0.2 | 70.5 | 2.96 ± 0.34 | 3.06 ± 0.29 | 50 |
| 3 Compound 75 | 9.7 ± 0.3# | 82.0 | 1.70 ± 0.26* | 1.50 ± 0.22 | 0 |
| 25 mg/kg PO | | | | | |
| 4 Compound 75 | 9.2 ± 0.1# | 77.9 | 2.11 ± 0.25* | 2.06 ± 0.28 | 22 |
| 50 mg/kg PO | | | | | |

PO-oral
DAI-Disease activity index
DAINWT-Disease activity index without the body weight parameter
*Statistically significant difference from Vehicle + DSS group p < 0.05 Mann-Whitney test
#Significant difference from Vehicle + DSS group p < 0.05 one way ANOVA and Turkey-Kramer Multiple Comparisons test

TABLE 2
EFFECT OF Compound 75 IN PERCENT WEIGHT CHANGE
FROM DSS-INDUCED COLITIS IN MICE

| | Mean Weight (grams) and % weight change (±SEM) | | | | | | |
|---|---|---|---|---|---|---|---|
| Treatment + DSS | | | % | | % | | % |
| Mg/kg | DAY 0 | DAY 5 | change | DAY 6 | change | DAY 7 | change |
| 1 Veh. PO no DSS | 25.2 ± 0.4 | 23.9 ± 0.4 | −5.2 ± 1.0 | 24.4 ± 0.5 | −3.2 ± 1.1 | 24.3 ± 0.5 | −3.6 ± 1.1 |
| 2 Veh. PO | 24.9 ± 0.4 | 23.4 ± 0.5 | −5.9 ± 2.2 | 23.5 ± 0.9 | −5.5 ± 3.6 | 21.9 ± 0.6 | −12.0 ± 2.6 |
| 3 Compound 75 | 25.1 ± 0.5 | 23.0 ± 0.5 | −8.3 ± 1.5 | 22.9 ± 0.7 | −8.7 ± 2.7 | 22.7 ± 0.9 | −9.6 ± 3.5 |
| 25 mg/kg PO | | | | | | | |
| 4 Compound 75 | 25.6 ± 0.5 | 23.1 ± 0.5 | −9.5 ± 1.3 | 23.3 ± 0.8 | −8.8 ± 2.2 | 23.2 ± 0.7 | −9.2 ± 1.9 |
| 50 mg/kg PO | | | | | | | |

PO-oral

In view of the above results the experiment was repeated with lower doses of compound 75. The above procedure was followed with the following groups

| GROUP NUMBER | PRODUCT ID | DOSE LEVEL (mg/kg) |
|---|---|---|
| 1 | Vehicle no DSS | 0 |
| 2 | Compound 75 with DSS | 0 |
| 3 | Compound 75 with DSS | 12.5 PO |
| 4 | Compound 75 with DSS | 25 PO |
| 5 | Compound 75 with DSS | 50 PO |

PO-oral

Individual doses were calculated based on daily body weights. Prior to administration, 50 mg of Compound 75 was diluted with 10 milliliters of 0.1N HCl (50 mg/kg for Group 5). The resulting solution was then further diluted twice, with 5 ml increments and used for the 25 (Group 4) and 12.5 (Group 3) mg/kg dose levels. The 0.1 N HCl was used for Groups 1 and 2 and constituted the vehicle control. Groups 2 through 5 were given a 5% w/w solution of dextran sulfate sodium (DSS) in distilled water substituted for their normal water supply (approximately 10 ml:mouse/day) from Days 0 through 7. Each group of animals received the appropriate amount of the test substance, as described above, by intubation using a stainless steel ball-tipped gavage needle attached to an appropriate syringe.

Results and Conclusion

In this study, DSS produced a typical degree of colonic inflammation and colonic shortening. However, there was not the significant loss of body weight observed in other studies. The use of 0.1 N HCl may have been a factor in the lack of weight loss. The use of the DAI, in which weight loss is an important component, underestimates the degree of colitis and therefore the disease activity index using presence of fecal blood and stool consistency (DAINWT) is probably a more reliable estimate of activity. The typical degree of colonic shortening was observed and this measure remains the most significant indication of the extent of colitis and the degree protection. Compound 75 produced a statistically significant protection at all doses based on DAI and DAINWT. The maximal effectiveness on prevention of colonic shortening was observed at the lowest dose tested (12.5 mg/kg PO daily). The degree of effectiveness on this compound compares favorably with other experimental compounds, which have been tested previously and also confirms the initial test done with twice a day dosing and administered in cyclodextrin. No deaths were observed during the duration of the experiment.

TABLE 1
EFFECT OF COMPOUND 75 AFTER PO ADMINISTRATION:
PREVENTION OF DSS-INDUCED COLITIS IN MICE

| | Mean | | | | |
|---|---|---|---|---|---|
| | Colon | % of | | | |
| Treatment | Length | Normal | DAI | | % Inh. |
| (mg/kg) + | Cm ± | Length | mean ± | DAINWT | Colonic |
| DSS | SEM | Shortening | SEM | mean ± SEM | Shortening |
| 1. Veh. PO no | 11.2 ± 0.2 | 100 | 0.11 ± 0.06* | 0.06 ± 0.06* | — |
| DSS | | | | | |
| 2. Veh. PO | 8.2 ± 0.2 | 73.1 | 2.26 ± 0.13 | 3.17 ± 0.12 | 0 |
| 3. Compound 75 | 10.0 ± 0.1# | 89.6 | 1.57 ± 0.17* | 2.25 ± 0.25* | 65 |
| 12.5 mg/kg PO | | | | | |
| 4. Compound 75 | 9.9 ± 0.3# | 88.7 | 1.53 ± 0.17* | 2.30 ± 0.25* | 58 |
| 25 mg/kg PO | | | | | |
| 5. Compound 75 | 9.7 ± 0.2# | 87.0 | 1.70 ± 0.18* | 2.30 ± 0.24* | 52 |
| 50 mg/kg PO | | | | | |

PO-oral
DAI-Disease Activity Index
DAINW-Disease Activity Index without the body weight parameter
*Statistically significant difference from Vehicle + DSS group p < 0.05 Mann-Whitney Test
#Significant difference from Vehicle + DSS group p < 0.05 one way ANOVA and Turkey-Kramer Multiple Comparisons Test

TABLE 2
EFFECT OF COMPOUND 75 IN PERCENT WEIGHT CHANGE
FROM DSS- INDUCED COLITIS IN MICE

| Treatment (mg/kg) + | | | | | | | |
|---|---|---|---|---|---|---|---|
| DSS | DAY 0 | DAY 5 | % change | DAY 6 | % change | DAY 7 | % change |
| 1. Veh. PO no | 23.8 ± 0.4 | 23.7 ± 0.6 | −0.5 ± 2.0 | 23.1 ± 0.5 | −2.8 ± 1.0 | 24.0 ± 0.5 | +0.9 ± 1.0 |
| DSS | | | | | | | |
| 2. Veh. PO | 24.6 ± 0.2 | 24.2 ± 0.4 | −1.3 ± 1.0 | 24.1 ± 0.3 | −1.8 ± 1.0 | 24.6 ± 0.4 | 0.0 ± 2.0 |
| 3. Compound 75 | 23.6 ± 0.3 | 23.9 ± 0.4 | +1.3 ± 1.0 | 23.6 ± 0.4 | 0.0 ± 1.0 | 23.9 ± 0.4 | +1.3 ± 1.0 |
| 12.5 mg/kg PO | | | | | | | |
| 4. Compound 75 | 24.2 ± 0.4 | 24.7 ± 0.6 | +2.0 ≅ 1.0 | 23.9 ± 0.5 | −1.2 ± 1.0 | 24.9 ± 0.5 | +2.9 ± 1.0 |
| 25 mg/kg PO | | | | | | | |
| 5. Compound 75 | 23.9 ± 0.3 | 23.9 ± 0.3 | −0.7 ± 1.0 | 23.5 ± 0.3 | −1.6 ± 1.0 | 23.8 ± 0.3 | −0.3 ± 2.0 |
| 50 mg/kg PO | | | | | | | |

PO-oral

Diagnostic Criteria for Inflammatory Bowel Disease

The diagnostic criteria for inflammatory bowel disease are those found in standard medical references e.g., Harrison's Principles of Internal Medicine, thirteenth ed., 1994, by McGraw-Hill, Inc., ISBN 0-07-032370-4, pgs. 1194-1197. These criteria may be used to determine when to begin using the method of the invention, the frequency and degree of treatment, and the time for cessation of treatment.

While the present invention has been illustrated above by certain specific embodiments, these are not intended to limit the scope of the invention as described in the appended claims.

1. Use of a farnesyl protein transferase inhibitor selected from the groups I, II, III, IV, V, VI, VII, VIII and IX infra for use in the manufacture of a medicament for the treatment of inflammatory bowel disease: 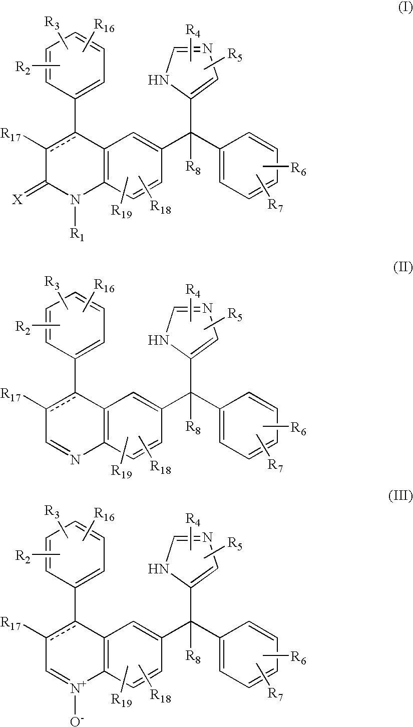
the pharmaceutically acceptable acid or base addition salts and the stereochemically isomeric forms thereof, wherein
the dotted line represents an optional bond;
X is oxygen or sulfur;
R¹ is hydrogen, C₁₋₁₂alkyl, Ar¹, Ar²C₁₋₆alkyl, quinolinylC₁₋₆alkyl, pyridylC₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl, or a radical of formula -Alk¹-C(═O)—R⁹, -Alk¹-S(O)—R⁹ or -Alk¹-S(O)₂—R⁹, whereinAlk¹ is C₁₋₆alkanediyl,
R⁹ is hydroxy, C₁₋₆alkyl, C₁₋₆alkyloxy, amino, C₁₋₈alkylamino or C₁₋₈alkylamino substituted with C₁₋₆alkyloxycarbonyl;
R², R³ and R¹⁶ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, aminoC₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl, 4,4-dimethyloxazolyl; or
when on adjacent positions R² and R³ taken together may form a bivalent radical of formula
—O—CH₂—O— (a-1),
—O—CH₂—CH₂—O— (a-2),
—O—CH═CH— (a-3),
—O—CH₂—CH₂— (a-4),
—O—CH₂—CH₂—CH₂— (a-5), or
—CH═CH—CH═CH— (a-6);
R⁴ and R⁵ each independently are hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;
R⁶ and R⁷ each independently are hydrogen, halo, cyano, C₁₋₆alkyl, C₁₋₆alkyloxy, Ar²oxy, trihalomethyl, C₁₋₆alkylthio, di(C₁₋₆alkyl)amino, or when on adjacent positions R⁶ and R⁷ taken together may form a bivalent radical of formula
—O—CH₂—O— (c-1), or
—CH═CH—CH═CH— (c-2);
R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, carboxyC₁₋₆alkyl, hydroxyC₁₋₆alkyl, aminoC₁₋₆alkyl, mono- or di(C₁₋₆alkyl)aminoC₁₋₆alkyl, imidazolyl, haloC₁₋₆alkyl, C₁₋₆alkyloxyC₁₋₆alkyl, aminocarbonylC₁₋₆alkyl, or a radical of formula
—O—R¹⁰ (b-1),
—S—R¹⁰ (b-2),
—N—R¹¹R¹² (b-3),
wherein R¹⁰ is hydrogen, C₁₋₆alkyl, C₁₋₆alkylcarbonyl, Ar¹, Ar²C₁₋₆alkyl, C₁₋₆alkyloxycarbonylC₁₋₆alkyl, or a radical or formula -Alk²-OR¹³ or -Alk²-NR¹⁴R¹⁵;
R¹¹ is hydrogen, C₁₋₁₂alkyl, Ar¹ or Ar²C₁₋₆alkyl;
R¹² is hydrogen, C₁₋₆alkyl, C₁₋₁₆alkylcarbonyl, C₁₋₆alkyloxycarbonyl, C₁₋₆alkylaminocarbonyl, Ar¹, Ar²C₁₋₆alkyl, C₁₋₆alkylcarbonylC₁₋₆alkyl, a natural amino acid, Ar¹carbonyl, Ar²C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical or formula -Alk²-OR¹³ or -Alk²-NR¹⁴R¹⁵;
wherein Alk² is C₁₋₆alkanediyl;
R¹³ is hydrogen, C₁₋₆alkyl, C₁₋₆alkylcarbonyl, hydroxyC₁₋₆alkyl, Ar¹ or Ar²C₁₋₆alkyl;
R¹⁴ is hydrogen, C₁₋₆alkyl, Ar¹ or Ar²C₁₋₆alkyl;
R¹⁵ is hydrogen, C₁₋₆alkyl, C₁₋₆alkylcarbonyl, Ar¹ or Ar²C₁₋₆alkyl;
R¹⁷ is hydrogen, halo, cyano, C₁₋₆alkyl, C₁₋₆alkyloxycarbonyl, Ar¹;
R¹⁸ is hydrogen, C₁₋₆alkyl, C₁₋₆alkyloxy or halo;
R¹⁹ is hydrogen or C₁₋₆alkyl;
Ar¹ is phenyl or phenyl substituted with C₁₋₆alkyl, hydroxy, amino, C₁₋₆alkyloxy or halo; and
Ar² is phenyl or phenyl substituted with C₁₋₆alkyl, hydroxy, amino, C₁₋₆alkyloxy or halo. 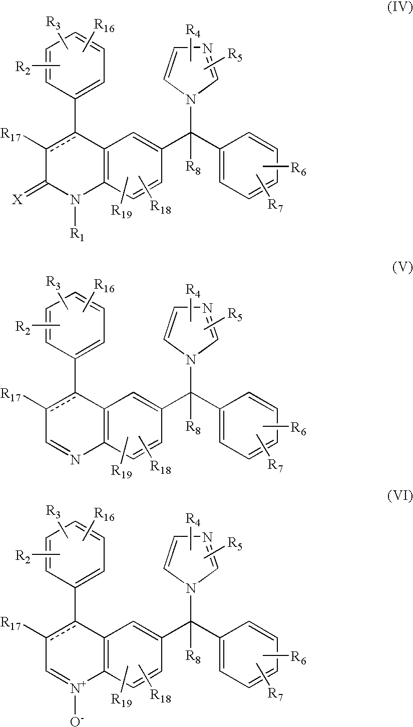
the pharmaceutically acceptable acid or base addition salts and the stereochemically isomeric forms thereof, wherein
the dotted line represents an optional bond;
X is oxygen or sulfur;
R¹ is hydrogen, C₁₋₁₂alkyl, Ar¹, Ar²C₁₋₆alkyl, quinolinylC₁₋₆alkyl, pyridylC₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl, or a radical of formula -Alk¹-C(═O)—R⁹, -Alk¹-S(O)—R⁹ or -Alk¹-S(O)₂—R⁹, whereinAlk¹ is C₁₋₆alkanediyl,
R⁹ is hydroxy, C₁₋₆alkyl, C₁₋₆alkyloxy, amino, C₁₋₈alkylamino or C₁₋₈alkylamino substituted with C₁₋₆alkyloxycarbonyl;
R² and R³ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, aminoC₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl; or
when on adjacent positions R² and R³ taken together may form a bivalent radical of formula
—O—CH₂—O— (a-1),
—O—CH₂—CH₂—O— (a-2),
—O—CH═CH— (a-3),
—O—CH₂—CH₂— (a-4),
—O—CH₂—CH₂—CH₂— (a-5), or
—CH═CH—CH═CH— (a-6);
R⁴ and R⁵ each independently are hydrogen, Ar¹, C₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;
R⁶ and R⁷ each independently are hydrogen, halo, cyano, C₁₋₆alkyl, C₁₋₆alkyloxy or Ar²oxy;
R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, hydroxycarbonylC₁₋₆alkyl, hydroxyC₁₋₆alkyl, aminoC₁₋₆alkyl, mono- or di(C₁₋₆alkyl)aminoC₁₋₆alkyl, haloC₁₋₆alkyl, C₁₋₆alkyloxyC₁₋₆alkyl, aminocarbonylC₁₋₆alkyl, Ar¹, Ar²C₁₋₆alkyloxyC₁₋₆alkyl, C₁₋₆alkylthioC₁₋₆alkyl;
R¹⁰ is hydrogen, C₁₋₆alkyl, C₁₋₆alkyloxy or halo;
R¹¹ is hydrogen or C₁₋₆alkyl;
Ar¹ is phenyl or phenyl substituted with C₁₋₆alkyl, hydroxy, amino, C₁₋₆alkyloxy or halo;
Ar² is phenyl or phenyl substituted with C₁₋₆alkyl, hydroxy, amino, C₁₋₆alkyloxy or halo. 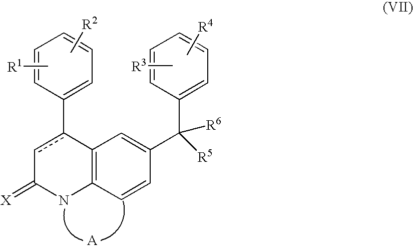
the pharmaceutically acceptable acid addition salts and the stereochemically isomeric forms thereof, wherein
the dotted line represents an optional bond;
X is oxygen or sulfur;
-A- is a bivalent radical of formula
—CH═CH— (a-1),
—CH₂—CH₂— (a-2),
—CH₂—CH₂—CH₂— (a-3),
—CH₂—O— (a-4),
—CH₂—CH₂—O— (a-5),
—CH₂—S— (a-6),
—CH₂—CH₂—S— (a-7),
—CH═N— (a-8),
—N═N— (a-9), or
—CO—NH— (a-10);
wherein optionally one hydrogen atom may be replaced by C₁₋₄alkyl or Ar¹;
R¹ and R² each independently are hydrogen, hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkyloxyC₁₋₆alkyloxy, C₁₋₆alkyloxycarbonyl, aminoC₁₋₆alkyloxy, mono- or di(C₁₋₆alkyl)aminoC₁₋₆alkyloxy, Ar², Ar²—C₁₋₆alkyl, Ar²-oxy, Ar²—C₁₋₆alkyloxy; or when on adjacent positions R¹ and R² taken together may form a bivalent radical of formula
—O—CH₂—O— (b-1),
—O—CH₂—CH₂—O— (b-2),
—O—CH═CH— (b-3),
—O—CH₂—CH₂— (b-4),
—O—CH₂—CH₂—CH₂— (b-5), or
—CH═CH—CH═CH— (b-6);
R³ and R⁴ each independently are hydrogen, halo, cyano, C₁₋₆alkyl, C₁₋₆alkyloxy, Ar³-oxy, C₁₋₆alkylthio, di(C₁₋₆alkyl)amino, trihalomethyl, trihalomethoxy, or
when on adjacent positions R³ and R⁴ taken together may form a bivalent radical of formula
—O—CH₂—O— (c-1),
—O—CH₂—CH₂—O— (c-2), or
—CH═CH—CH═CH— (c-3);
R⁵ is a radical of formula 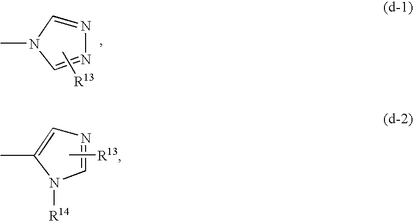
wherein R¹³ is hydrogen, halo, Ar⁴, C₁₋₆alkyl, hydroxyC₁₋₆alkyl, C₁₋₆alkyloxyC₁₋₆alkyl, C₁₋₆alkyloxy, C₁₋₆alkylthio, amino, C₁₋₆alkyloxycarbonyl, C₁₋₆alkylS(O)C₁₋₆alkyl or C₁₋₆alkylS(O)₂C₁₋₆alkyl;
R¹⁴ is hydrogen, C₁₋₆alkyl or di(C₁₋₄alkyl)aminosulfonyl;
R⁶ is hydrogen, hydroxy, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, C₁₋₆alkyloxycarbonylC₁₋₆alkyl, C₁₋₆alkylcarbonyl-C₁₋₆alkyl, C₁₋₆alkyloxycarbonyl, mono- or di(C₁₋₆alkyl)aminoC₁₋₆alkyl, Ar⁵, Ar⁵-C₁₋₆alkyloxyC₁₋₆alkyl; or a radical of formula
—O—R⁷ (e-1),
—S—R⁷ (e-2),
—N—R⁸R⁹ (e-3),
wherein R⁷ is hydrogen, C₁₋₆alkyl, C₁₋₆alkylcarbonyl, Ar⁶, Ar⁶—C₁₋₆alkyl, C₁₋₆alkyloxycarbonylC₁₋₆alkyl, or a radical of formula -Alk-OR¹⁰ or -Alk-NR¹¹R¹²;
R⁸ is hydrogen, C₁₋₆alkyl, Ar⁷ or Ar⁷—C₁₋₆alkyl;
R⁹ is hydrogen, C₁₋₆alkyl, C₁₋₆alkylcarbonyl, C₁₋₆alkyloxycarbonyl, C₁₋₆alkylaminocarbonyl, Ar⁸, Ar⁸—C₁₋₆alkyl, C₁₋₆alkylcarbonyl-C₁₋₆alkyl, Ar⁸-carbonyl, Ar⁸—C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical or formula -Alk-OR¹⁰ or -Alk-NR¹¹R¹²;
wherein Alk is C₁₋₆alkanediyl;
R¹⁰ is hydrogen, C₁₋₆alkyl, C₁₋₆alkylcarbonyl, hydroxyC₁₋₆alkyl, Ar⁹ or Ar⁹—C₁₋₆alkyl;
R¹¹ is hydrogen, C₁₋₆alkyl, C₁₋₆alkylcarbonyl, Ar¹⁰ or Ar¹⁰—C₁₋₆alkyl;
R¹² is hydrogen, C₁₋₆alkyl, Ar¹¹ or Ar¹¹—C₁₋₆alkyl; and
Ar¹ to Ar¹¹ are each independently selected from phenyl; or phenyl substituted with halo, C₁₋₆alkyl, C₁₋₆alkyloxy or trifluoromethyl. 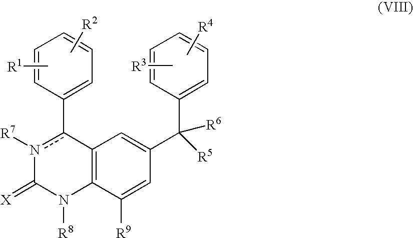
the pharmaceutically acceptable acid addition salts and the stereochemically isomeric forms thereof, wherein
the dotted line represents an optional bond;
X is oxygen or sulfur;
R¹ and R² each independently are hydrogen, hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkyloxyC₁₋₆alkyloxy, C₁₋₆alkyloxycarbonyl, aminoC₁₋₆alkyloxy, mono- or di(C₁₋₆alkyl)aminoC₁₋₆alkyloxy, Ar¹, Ar¹C₁₋₆alkyl, Ar¹oxy or Ar¹C₁₋₆alkyloxy;
R³ and R⁴ each independently are hydrogen, halo, cyano, C₁₋₆alkyl, C₁₋₆alkyloxy, Ar¹oxy, C₁₋₆alkylthio, di(C₁₋₆alkyl)amino, trihalomethyl or trihalomethoxy;
R⁵ is hydrogen, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, C₁₋₆alkyloxycarbonylC₁₋₆alkyl, C₁₋₆alkylcarbonyl-C₁₋₆alkyl, C₁₋₆alkyloxycarbonyl, mono- or di(C₁₋₆alkyl)aminoC₁₋₆alkyl, Ar¹, Ar¹ C₁₋₆alkyloxyC₁₋₆alkyl; or a radical of formula
—O—R¹⁰ (a-1),
—S—R¹⁰ (a-2),
—N—R¹¹R¹² (a-3),
wherein R¹⁰ is hydrogen, C₁₋₆alkyl, C₁₋₆alkylcarbonyl, Ar¹, Ar¹ C₁₋₆alkyl, C₁₋₆alkyloxycarbonylC₁₋₆alkyl, or a radical of formula -Alk-OR¹³ or -Alk-NR¹⁴R¹⁵;
R¹¹ is hydrogen, C₁₋₆alkyl, Ar¹ or Ar¹C₁₋₆alkyl;
R¹² is hydrogen, C₁₋₆alkyl, C₁₋₆alkylcarbonyl, C₁₋₆alkyloxycarbonyl, C₁₋₆alkylaminocarbonyl, Ar¹, Ar¹C₁₋₆alkyl, C₁₋₆alkylcarbonyl-C₁₋₆alkyl, Ar¹carbonyl, Ar¹C₁₋₆alkylcarbonyl, aminocarbonylcarbonyl, C₁₋₆alkyloxyC₁₋₆alkylcarbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical or formula -Alk-OR¹³ or -Alk-NR¹⁴R¹⁵;
whereinAlk is C₁₋₆alkanediyl;
R¹³ is hydrogen, C₁₋₆alkyl, C₁₋₆alkylcarbonyl, hydroxyC₁₋₆alkyl, Ar¹ or Ar¹C₁₋₆alkyl;
R¹⁴ is hydrogen, C₁₋₆alkyl, Ar¹ or Ar¹C₁₋₆alkyl;
R¹⁵ is hydrogen, C₁₋₆alkyl, C₁₋₆alkylcarbonyl, Ar¹ or Ar¹C₁₋₆alkyl;
R⁶ is a radical of formula 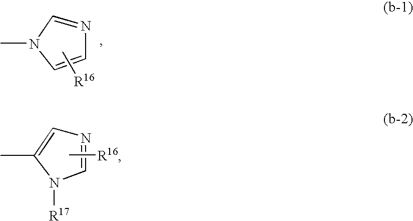
wherein R¹⁶ is hydrogen, halo, Ar¹, C₁₋₆alkyl, hydroxyC₁₋₆alkyl, C₁₋₆alkyloxyC₁₋₆alkyl, C₁₋₆alkyloxy, C₁₋₆alkylthio, amino, C₁₋₆alkyloxycarbonyl, C₁₋₆alkylthioC₁₋₆alkyl, C₁₋₆alkylS(O)C₁₋₆alkyl or C₁₋₆alkylS(O)₂C₁₋₆alkyl;
R¹⁷ is hydrogen, C₁₋₆alkyl or di(C₁₋₄alkyl)aminosulfonyl;
R⁷ is hydrogen or C₁₋₆alkyl provided that the dotted line does not represent a bond;
R⁸ is hydrogen, C₁₋₆alkyl or Ar²CH₂ or Het¹ CH₂;
R⁹ is hydrogen, C₁₋₆alkyl, C₁₋₆alkyloxy or halo; or
R⁸ and R⁹ taken together to form a bivalent radical of formula
—CH═CH— (c-1),
—CH₂—CH₂— (c-2),
—CH₂—CH₂—CH₂— (c-3),
—CH₂—O— (c-4), or
—CH₂—CH₂—O— (c-5);
Ar¹ is phenyl; or phenyl substituted with 1 or 2 substituents each independently selected from halo, C₁₋₆alkyl, C₁₋₆alkyloxy or trifluoromethyl;
Ar² is phenyl; or phenyl substituted with 1 or 2 substituents each independently selected from halo, C₁₋₆alkyl, C₁₋₆alkyloxy or trifluoromethyl; and
Het¹ is pyridinyl; pyridinyl substituted with 1 or 2 substituents each independently selected from halo, C₁₋₆alkyl, C₁₋₆alkyloxy or trifluoromethyl
and 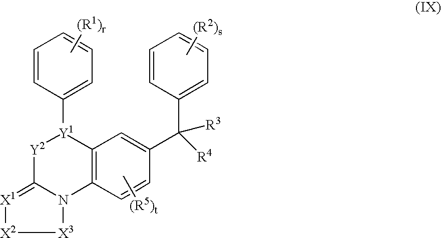
or the pharmaceutically acceptable acid addition salts and the stereochemically isomeric forms thereof, wherein
═X¹—X²—X³— is a trivalent radical of formula
═N—CR⁶═CR⁷— (x-1),
═N—N═CR⁶— (x-2),
═N—NH—C(═O)— (x-3),
═N—N═N— (x-4),
═N—CR⁶═N— (x-5),
═CR⁶—CR⁷═CR⁸— (x-6),
═CR⁶—N═CR⁷— (x-7),
═CR⁶—NH—C(═O)— (x-8), or
═CR⁶—N═N— (x-9);
wherein each R⁶, R⁷ and R⁸ are independently hydrogen, C₁₋₄alkyl, hydroxy, C₁₋₄alkyloxy, aryloxy, C₁₋₄alkyloxycarbonyl, hydroxyC₁₋₄alkyl, C₁₋₄alkyloxyC₁₋₄alkyl, mono- or di(C₁₋₄alkyl)aminoC₁₋₄alkyl, cyano, amino, thio, C₁₋₄alkylthio, arylthio or aryl;
>Y¹—Y²— is a trivalent radical of formula
>CH—CHR⁹— (y-1),
>C═N— (y-2),
>CH—NR⁹— (y-3), or
>C═CR⁹— (y-4);
wherein each R⁹ independently is hydrogen, halo, halocarbonyl, aminocarbonyl, hydroxyC₁₋₄alkyl, cyano, carboxyl, C₁₋₄alkyl, C₁₋₄alkyloxy, C₁₋₄alkyloxyC₁₋₄alkyl, C₁₋₄alkyloxycarbonyl, mono- or di(C₁₋₄alkyl)amino, mono- or di(C₁₋₄alkyl)aminoC₁₋₄alkyl, aryl;
r and s are each independently 0, 1, 2, 3, 4 or 5;
t is 0, 1, 2 or 3;
each R¹ and R² are independently hydroxy, halo, cyano, C₁₋₆alkyl, trihalomethyl, trihalomethoxy, C₂₋₆alkenyl, C₁₋₆alkyloxy, hydroxyC₁₋₆alkyloxy, C₁₋₆alkylthio, C₁₋₆alkyloxyC₁₋₆alkyloxy, C₁₋₆alkyloxycarbonyl, aminoC₁₋₆alkyloxy, mono- or di(C₁₋₆alkyl)amino, mono- or di(C₁₋₆alkyl)aminoC₁₋₆alkyloxy, aryl, arylC₁₋₆alkyl, aryloxy or arylC₁₋₆alkyloxy, hydroxycarbonyl, C₁₋₆alkyloxycarbonyl, aminocarbonyl, aminoC₁₋₆alkyl, mono- or di(C₁₋₆alkyl)aminocarbonyl, mono- or di(C₁₋₆alkyl)aminoC₁₋₆alkyl; or
two R¹ or R² substituents adjacent to one another on the phenyl ring may independently form together a bivalent radical of formula
—O—CH₂—O— (a-1),
—O—CH₂—CH₂—O— (a-2),
—O═CH═CH— (a-3),
—O—CH₂—CH₂— (a-4),
—O—CH₂—CH₂—CH₂— (a-5), or
—CH═CH—CH═CH— (a-6);
R³ is hydrogen, halo, C₁₋₆alkyl, cyano, haloC₁₋₆alkyl, hydroxyC₁₋₆alkyl, cyanoC₁₋₆alkyl, aminoC₁₋₆alkyl, C₁₋₆alkyloxyC₁₋₆alkyl, C₁₋₆alkylthioC₁₋₆alkyl, aminocarbonylC₁₋₆alkyl, hydroxycarbonyl, hydroxycarbonylC₁₋₆alkyl, C₁₋₆alkyloxycarbonylC₁₋₆alkyl, C₁₋₆alkylcarbonylC₁₋₆alkyl, C₁₋₆alkyloxycarbonyl, aryl, arylC₁₋₆alkyloxyC₁₋₆alkyl, mono- or di(C₁₋₆alkyl)aminoC₁₋₆alkyl;
or a radical of formula
—O—R¹⁰ (b-1),
—S—R¹⁰ (b-2),
—NR¹¹R¹² (b-3),
wherein R¹⁰ is hydrogen, C₁₋₆alkyl, C₁₋₆alkylcarbonyl, aryl, arylC₁₋₆alkyl, C₁₋₆alkyloxycarbonylC₁₋₆alkyl, or a radical of formula -Alk-OR¹³ or -Alk-NR¹⁴R¹⁵;
R¹¹ is hydrogen, C₁₋₆alkyl, aryl or arylC₁₋₆alkyl;
R¹² is hydrogen, C₁₋₆alkyl, aryl, hydroxy, amino, C₁₋₆alkyloxy, C₁₋₆alkylcarbonylC₁₋₆alkyl, arylC₁₋₆alkyl, C₁₋₆alkylcarbonylamino, mono- or di(C₁₋₆alkyl)amino, C₁₋₆alkylcarbonyl, aminocarbonyl, arylcarbonyl, haloC₁₋₆alkylcarbonyl, arylC₁₋₆alkylcarbonyl, C₁₋₆alkyloxycarbonyl, C₁₋₆alkyloxyC₁₋₆alkylcarbonyl, mono- or di(C₁₋₆alkyl)aminocarbonyl wherein the alkyl moiety may optionally be substituted by one or more substituents independently selected from aryl or C₁₋₃alkyloxycarbonyl, aminocarbonylcarbonyl, mono- or di(C₁₋₆alkyl)aminoC₁₋₆alkylcarbonyl, or a radical or formula -Alk-OR¹³ or -Alk-NR¹⁴R¹⁵;
wherein Alk is C₁₋₆alkanediyl;
R¹³ is hydrogen, C₁₋₆alkyl, C₁₋₆alkylcarbonyl, hydroxyC₁₋₆alkyl, aryl or arylC₁₋₆alkyl;
R¹⁴ is hydrogen, C₁₋₆alkyl, aryl or arylC₁₋₆alkyl;
R¹⁵ is hydrogen, C₁₋₆alkyl, C₁₋₆alkylcarbonyl, aryl or arylC₁₋₆alkyl;
R⁴ is a radical of formula 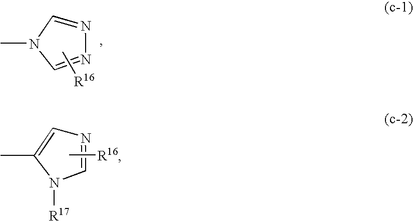
wherein R¹⁶ is hydrogen, halo, aryl, C₁₋₆alkyl, hydroxyC₁₋₆alkyl, C₁₋₆alkyloxyC₁₋₆alkyl, C₁₋₆alkyloxy, C₁₋₆alkylthio, amino, mono- or di(C₁₋₄alkyl)amino, hydroxycarbonyl, C₁₋₆alkyloxycarbonyl, C₁₋₆alkylthioC₁₋₆alkyl, C₁₋₆alkylS(O)C₁₋₆alkyl or C₁₋₆alkylS(O)₂C₁₋₆alkyl;
R¹⁶ may also be bound to one of the nitrogen atoms in the imidazole ring of formula (c-1) or (c-2), in which case the meaning of R¹⁶ when bound to the nitrogen is limited to hydrogen, aryl, C₁₋₆alkyl, hydroxyC₁₋₆alkyl, C₁₋₆alkyloxyC₁₋₆alkyl, C₁₋₆alkyloxycarbonyl, C₁₋₆alkylS(O)C₁₋₆alkyl or C₁₋₆alkylS(O)₂C₁₋₆alkyl;
R¹⁷ is hydrogen, C₁₋₆alkyl, C₁₋₆alkyloxyC₁₋₆alkyl, arylC₁₋₆alkyl, trifluoromethyl or di(C₁₋₄alkyl)aminosulfonyl;
R⁵ is C₁₋₆alkyl, C₁₋₆alkyloxy or halo; aryl is phenyl, naphthalenyl or phenyl substituted with 1 or more substituents each independently selected from halo, C₁₋₆alkyl, C₁₋₆alkyloxy or trifluoromethyl.

2. Use according to claim 1 wherein said farnesyl protein transferase inhibitor is a compound of formula (I), or a compound of formula (II) or (III) which is metabolized in vivo to a compound of formula (I), said compounds being represent by 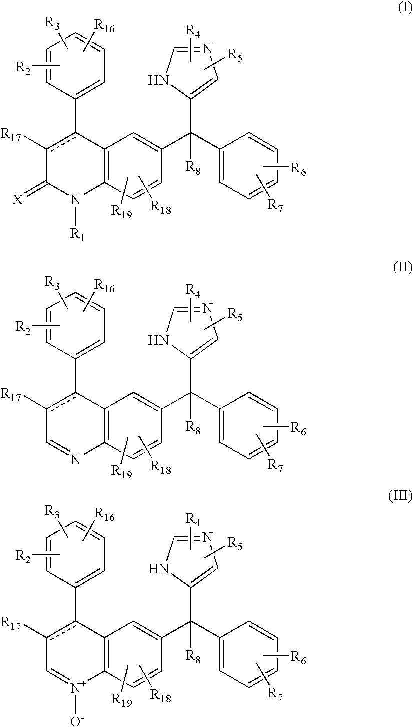
a stereoisomeric form thereof, a pharmaceutically acceptable acid or base addition salt thereof, wherein
the dotted line represents an optional bond;
X is oxygen or sulfur;
R¹ is hydrogen, C₁₋₁₂alkyl, Ar¹, Ar²C₁₋₆alkyl, quinolinylC₁₋₆alkyl, pyridylC₁₋₆alkyl, hydroxyC₁₋₆alkyl, C₁₋₆alkyloxyC₁₋₆alkyl, mono- or di(C₁₋₆alkyl)aminoC₁₋₆alkyl, aminoC₁₋₆alkyl, or a radical of formula -Alk¹-C(═O)—R⁹, -Alk¹-S(O)—R⁹ or -Alk¹-S(O)₂—R⁹, whereinAlk¹ is C₁₋₆alkanediyl,
R⁹ is hydroxy, C₁₋₆alkyl, C₁₋₆alkyloxy, amino, C₁₋₈alkylamino or C₁₋₈alkylamino substituted with C₁₋₆alkyloxycarbonyl;
R², R³ and R¹⁶ each independently are hydrogen, hydroxy, halo, cyano, C₁₋₆alkyl, C₁₋₆alkyloxy, hydroxyC₁₋₆alkyloxy, C₁₋₆alkyloxyC₁₋₆alkyloxy, amino-C₁₋₆alkyloxy, mono- or di(C₁₋₆alkyl)aminoC₁₋₆alkyloxy, Ar¹, Ar²C₁₋₆alkyl, Ar²oxy, Ar²C₁₋₆alkyloxy, hydroxycarbonyl, C₁₋₆alkyloxycarbonyl, trihalomethyl, trihalomethoxy, C₂₋₆alkenyl, 4,4-dimethyloxazolyl; or when on adjacent positions R² and R³ taken together may form a bivalent radical of formula
—O—CH₂—O— (a-1),
—O—CH₂—CH₂—O— (a-2),
—O—CH═CH— (a-3),
—O—CH₂—CH₂— (a-4),
—O—CH₂—CH₂—CH₂— (a-5), or
—CH═CH—CH═CH— (a-6);
R⁴ and R⁵ each independently are hydrogen, halo, Ar¹, C₁₋₆alkyl, hydroxy-C₁₋₆alkyl, C₁₋₆alkyloxyC₁₋₆alkyl, C₁₋₆alkyloxy, C₁₋₆alkylthio, amino, hydroxycarbonyl, C₁₋₆alkyloxycarbonyl, C₁₋₆alkylS(O)C₁₋₆alkyl or C₁₋₆alkylS(O)₂C₁₋₆alkyl;
R⁶ and R⁷ each independently are hydrogen, halo, cyano, C₁₋₆alkyl, C₁₋₆alkyloxy, Ar²oxy, trihalomethyl, C₁₋₆alkylthio, di(C₁₋₆alkyl)amino, or
when on adjacent positions R⁶ and R⁷ taken together may form a bivalent radical of formula
—O—CH₂—O— (c-1), or
—CH═CH—CH═CH— (c-2);
R⁸ is hydrogen, C₁₋₆alkyl, cyano, hydroxycarbonyl, C₁₋₆alkyloxycarbonyl, C₁₋₆alkylcarbonylC₁₋₆alkyl, cyanoC₁₋₆alkyl, C₁₋₆alkyloxycarbonylC₁₋₆alkyl, carboxyC₁₋₆alkyl, hydroxyC₁₋₆alkyl, aminoC₁₋₆alkyl, mono- or di(C₁₋₆alkyl)-aminoC₁₋₆alkyl, imidazolyl, haloC₁₋₆alkyl, C₁₋₆alkyloxy-C₁₋₆alkyl, aminocarbonylC₁₋₆alkyl, or a radical of formula
—O—R¹⁰ (b-1),
—S—R¹⁰ (b-2),
—N—R¹¹R¹² (b-3),
wherein R¹⁰ is hydrogen, C₁₋₆alkyl, C₁₋₆alkylcarbonyl, Ar¹, Ar²C₁₋₆alkyl, C₁₋₆alkyloxycarbonylC₁₋₆alkyl, a radical or formula -Alk²-OR¹³ or -Alk²-NR¹⁴R¹⁵;
R¹¹ is hydrogen, C₁₋₁₂alkyl, Ar¹ or Ar²C₁₋₆alkyl;
R¹² is hydrogen, C₁₋₆alkyl, C₁₋₁₆alkylcarbonyl, C₁₋₆alkyloxy-carbonyl, C₁₋₆alkylaminocarbonyl, Ar¹, Ar²C₁₋₆alkyl, C₁₋₆alkylcarbonylC₁₋₆alkyl, a natural amino acid, Ar¹carbonyl, Ar²C₁₋₆alkylcarbonyl, aminocarbonylcarbonyl, C₁₋₆alkyloxy-C₁₋₆alkyl-carbonyl, hydroxy, C₁₋₆alkyloxy, aminocarbonyl, di(C₁₋₆alkyl)aminoC₁₋₆alkylcarbonyl, amino, C₁₋₆alkylamino, C₁₋₆alkylcarbonylamino, or a radical of formula -Alk²-OR¹³ or -Alk²-NR¹⁴R¹⁵; whereinAlk² is C₁₋₆alkanediyl;
R¹³ is hydrogen, C₁₋₆alkyl, C₁₋₆alkylcarbonyl, hydroxyC₁₋₆alkyl, Ar¹ or Ar²C₁₋₆alkyl;
R¹⁴ is hydrogen, C₁₋₆alkyl, Ar¹ or Ar²C₁₋₆alkyl;
R¹⁵ is hydrogen, C₁₋₆alkyl, C₁₋₆alkylcarbonyl, Ar¹ or Ar²C₁₋₆alkyl;
R¹⁷ is hydrogen, halo, cyano, C₁₋₆alkyl, C₁₋₆alkyloxycarbonyl, Ar¹;
R¹⁸ is hydrogen, C₁₋₆alkyl, C₁₋₆alkyloxy or halo;
R¹⁹ is hydrogen or C₁₋₆alkyl;
Ar¹ is phenyl or phenyl substituted with C₁₋₆alkyl, hydroxy, amino, C₁₋₆alkyloxy or halo; and
Ar² is phenyl or phenyl substituted with C₁₋₆alkyl, hydroxy, amino, C₁₋₆alkyloxy or halo.

3. Use according to claim 2 wherein said farnesyl protein transferase inhibitor is a compound of formula (I) and wherein R⁸ is hydrogen, hydroxy, haloC₁₋₆alkyl, hydroxyC₁₋₆alkyl, cyanoC₁₋₆alkyl, C₁₋₆alkyloxycarbonylC₁₋₆alkyl, imidazolyl, or a radical of formula —NR¹¹R¹² wherein R¹¹ is hydrogen or C₁₋₁₂alkyl and R¹² is hydrogen, C₁₋₆alkyl, C₁₋₆alkyloxy, C₁₋₆alkyloxyC₁₋₆alkylcarbonyl, hydroxy, or a radical of formula -Alk²-OR¹³ wherein R¹³ is hydrogen or C₁₋₆alkyl.

4. Use according to claim 1 wherein the compound is
4-(3-chlorophenyl)-6-[(4-chlorophenyl)hydroxy(1-methyl-1H-imidazol-5-yl)methyl]-1-methyl-2(1H)-quinolinone,
6-[amino(4-chlorophenyl)-1-methyl-1H-imidazol-5-ylmethyl]-4-(3-chlorophenyl)-1-methyl-2(1H)-quinolinone;
6-[(4-chlorophenyl)hydroxy(1-methyl-1H-imidazol-5-yl)methyl]-4-(3-ethoxyphenyl)-1-methyl-2(1H)-quinolinone;
6-[(4-chlorophenyl)(1-methyl-1H-imidazol-5-yl)methyl]-4-(3-ethoxyphenyl)-1-methyl-2(1H)-quinolinone monohydrochloride.monohydrate;
6-[amino(4-chlorophenyl)(1-methyl-1H-imidazol-5-yl)methyl]-4-(3-ethoxyphenyl)-1-methyl-2(1H)-quinolinone, and
6-amino(4-chlorophenyl)(1-methyl-1H-imidazol-5-yl)methyl]-1-methyl-4-(3-propylphenyl)-2(1H)-quinolinone; a stereoisomeric form thereof or a stereoisomeric form or a pharmaceutically acceptable acid or base addition salt thereof,

5. Use according to claim 1 wherein the compound is
(+)-6-[amino(4-chlorophenyl)(1-methyl-1H-imidazol-5-yl)methyl]-4-(3-chlorophenyl)-1-methyl-2(1H)-quinolinone; or a pharmaceutically acceptable acid addition salt thereof.

6. Use according to any of claims 1 to 5 for the treatment of Crohn's disease.

7. Use according to any of claims 1 to 5 for the treatment of ulcerative colitis.

8. Use according to any of claims 1 to 7 wherein the medicament is adapted for oral, rectal or parenteral administration.

9. A method of treating inflammatory bowel disease in a mammal comprising administering a therapeutically effective amount of a farnesyl protein transferase inhibitor described in any of claims 1 to 5 to said mammal.

10. A method of preventing inflammatory bowel disease in a mammal comprising administering a therapeutically effective amount of a farnesyl protein transferase inhibitor described in any of claims 1 to 5 to said mammal.

11. A pharmaceutical combination comprising a farnesyl transferase inhibitor as described in any claims 1 to 5 and one or more inflammatory bowel disease treatment agents.
